(19) United States
(12) Patent Application Publication
(10) Pub. No.: US 2008/0161249 A1
(43) Pub. Date: Jul. 3, 2008

(54) Use of Novel Antibacterial Compounds

(75) Inventor: Jianzhong Huang, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION, Philadelphia, PA (US)

(21) Appl. No.: 11/718,400

(22) PCT Filed: Nov. 17, 2005
(86) PCT No.: PCT/US05/41591

§ 371 Date: May 2, 2007

Related U.S. Application Data
(60) Provisional application No. 60/628,747, filed on Nov. 17, 2004.

(51) Int. Cl. A61K 31/7048; A61K 31/505; A61K 31/5377; A61P 31/04; A61K 31/53

(52) U.S. Cl. 514/29; 514/275; 514/235.8; 514/243; 514/30; 514/28

(57) ABSTRACT

The present invention relates to the use of novel antibacterial compounds, and pharmaceutical compositions containing these compounds.

### FIELD OF THE INVENTION

The present invention relates to the use of novel antibacterial compounds, and pharmaceutical compositions containing these compounds.

### BACKGROUND OF THE INVENTION

Bacterial initiator methionyl tRNA is modified by methionyl tRNA formyltransferase (FMT) to produce formyl-methionyl tRNA. Formyl methionine (f-met) is therefore present at the N-termini of all newly synthesized polypeptides. Polypeptide deformylase (PDF) then deformylates primary translation products to produce N-methionyl polypeptides. Most intracellular proteins are further processed by methionine amino peptidase (MAP) to yield the mature peptide and free methionine, which is recycled. PDF and MAP are both essential for bacterial growth, and PDF is required for MAP activity. This series of reactions is referred to as the methionine cycle (FIG. 1).

To date, polypeptide deformylase homologous proteins have been found in bacteria, in chloroplast-containing plants, as well as in mouse and human mitochondria. The eukaryotic proteins are nuclear encoded but carry a chloroplast/mitochondria localisation signal. This is consistent with the observation that chloroplast/mitochondria RNA and protein synthesis processes are highly similar to those of eubacteria. While it has been proposed that deformylation is an essential function in the chloroplasts of higher plants (Serero, T., Giglione, C. and Meinnel, T. (2001). J. Mol. Biol., 314, 695-708), the information available to date shows that human mitochondrial PDF (Bayer Aktiengesellschaft, Pat. WO2001/42431) is not as active as its bacterial counterparts and its functional role in normal human cells, if any, has not been demonstrated (Nguyen, K. T., Xubo, H., Colton, C., Chakrabarti, R., Zhu, M. X. and Pei, D. (2003). Biochemistry, 42, 9952-9958; Serero, A., Giglione, C., Sardini, A., Martinez-Sanz, J. and Meinnel, T. (2003). J. Biol. Chem., 278, 52953-52963).

Polypeptide deformylase is found in all eubacteria for which high coverage genomic sequence information is available. Sequence diversity among PDF homologs is high, with as little as 20% identity between distantly related proteins. However, conservation around the active site is very high, with several completely conserved residues, including one cysteine and two histidines which are required to coordinate the active site metal (Meinnel, T. et al., J. Mol. Biol. 267, 749-761, 1997).

PDF is recognized to be an attractive antibacterial target, as this enzyme has been demonstrated to be essential for bacterial growth in vitro (Mazel, D. et al., EMBO J. 13 (4), 914-923, 1994), is not believed to be involved in eukaryotic protein synthesis (Rajagopalan et al., J. Am. Chem. Soc. 119, 12418-12419, 1997), and is universally conserved in prokaryotes (Kozak, M., Microbiol. Rev. 47, 1-45, 1983). Therefore PDF inhibitors can potentially serve as broad spectrum antibacterial agents.

Surprisingly the present inventors have discovered that co-administration of a PDF inhibitor and a macrolide antibiotic results in a synergistic effect. In other words, the MIC of PDF inhibitors is reduced in the presence of subinhibitory concentrations of macrolide antibiotics, and vice versa, i.e. subinhibitory concentrations of PDF inhibitors decrease the MIC of macrolide antibiotics. The net result is that when a macrolide antibiotic and a PDF inhibitor are co-administered, much less concentration of both is needed to achieve the same anti-bacterial effect obtained by one agent (i.e. macrolide antibiotic or PDF inhibitor alone). Furthermore, the synergistic effect occurs for all pathogens, including, but not limited to, the genera of Streptococcus, Staphylococcus, Mycoplasma, Mycobacterium, Haemophilus, Moraxella, Escherichia, Salmonella, Klebsiella, Legionella, Chlamydia, Pseudomonas, Helicobacter, Neisseria, Proteus, Yersinia, Brucella, Borrelia, Treponema, Enterobacter, and Bordetella, and in particular, Streptococcus pneumoniae, Staphylococcus aureus and Haemophilus influenzae.

### SUMMARY OF THE INVENTION

The present invention involves co-administration of a PDF inhibitor (or a pharmaceutically acceptable salt, solvate, or physiologically functional derivative thereof) and a macrolide antibiotic (or a pharmaceutically acceptable salt, solvate, or physiologically functional derivative thereof).

In another aspect, the invention relates to a pharmaceutical composition comprising a PDF inhibitor (or a pharmaceutically acceptable salt, solvate, or physiologically functional derivative thereof) and a macrolide antibiotic (or a pharmaceutically acceptable salt, solvate, or physiologically functional derivative thereof).

### BRIEF DESCRIPTION OF THE DRAWING

FIG. 1 is the methionine cycle.

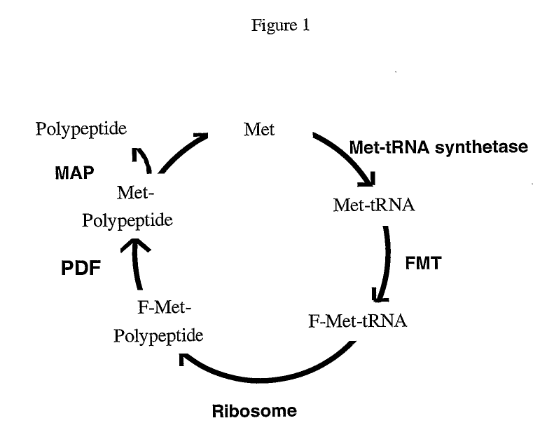

### DETAILED DESCRIPTION OF THE INVENTION

The present inventors have discovered that a PDF inhibitor when co-administered with a macrolide antibiotic can elicit a synergistic effect. In other words, the MIC of PDF inhibitors is reduced in the presence of subinhibitory concentrations of macrolide antibiotics, and vice versa, i.e. subinhibitory concentrations of PDF inhibitors decrease the MIC of macrolide antibiotics. The net result is that when a macrolide antibiotic and a PDF inhibitor are co-administered, much less concentration of both is needed to achieve the same anti-bacterial effect obtained by one agent (i.e. macrolide antibiotic or PDF inhibitor alone). Such a synergistic effect occurs for all pathogens. Thus the present invention involves co-administration of a PDF inhibitor (or a pharmaceutically acceptable salt, solvate, or physiologically functional derivative thereof) and a macrolide antibiotic (or a pharmaceutically acceptable salt, solvate, or physiologically functional derivative thereof).

In another aspect, the invention relates to a pharmaceutical composition comprising a PDF inhibitor (or a pharmaceutically acceptable salt, solvate, or physiologically functional derivative thereof) and a macrolide antibiotic (or a pharmaceutically acceptable salt, solvate, or physiologically functional derivative thereof).

Any PDF inhibitors can be used as embodiment of this invention. However, in one preferred embodiment, the PDF inhibitors are compounds of formula (1) as described in WO 2003101442, published Dec. 11, 2003:

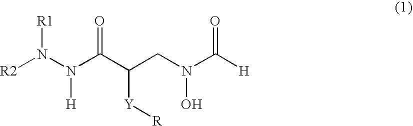

wherein R, R1, R2, are Y are as defined in WO2003101442, i.e.,
R is selected from the group consisting of:
C₂₋₆ alkyl (optionally substituted by alkoxy, halogen, or C₁₋₃ alkylsulfanyl); C₂₋₆ alkenyl (optionally substituted by alkoxy, halogen, or C₁₋₃ alkylsulfanyl); C₂₋₆ alkynyl (optionally substituted by alkoxy, halogen, or C₁₋₃ alkylsulfanyl); (CH₂)ₙ—C₃₋₆ carbocycle (optionally substituted by alkoxy, halogen, or C₁₋₃ alkylsulfanyl); and (CH₂)ₙ—R4, wherein R4 is selected from the group consisting of phenyl, furan, benzofuran, thiophene, benzothiophene, tetrahydrofuran, tetrahydropyran, dioxane, 1,4-benzodioxane or benzo[1,3]dioxole; R4 is optionally substituted by one or more substituent selected from Cl, Br, I, C₁₋₃ alkyl (optionally substituted by one to three F) and C₁₋₂ alkoxy (optionally substituted by one to three F);
   R1 and R2 are independently selected from the group consisting of:
hydrogen, C₁₋₃ substituted alkyl, C₂₋₃ substituted alkenyl, C₂₋₃ substituted alkynyl, (CH₂)ₙ—C₃₋₆ substituted carbocycle, aryl, heteroaryl, and heterocyclic;
   Y represents O, CH₂ or a covalent bond; and
   n is an integer from 0 to 2,
   or a salt, solvate, or physiologically functional derivative thereof.

Moreover, in a compound of formula (1), the most preferred R2 group is hydrogen. The most preferred absolute configuration of compounds of the formula (1) is indicated below:

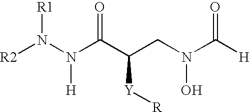

Even more preferred PDF inhibitors useful in the present invention within the meaning of a compound of formula (1) are selected from the group consisting of:
N-Hydroxy-N—[(R)-2-(N′-pyridin-2-yl-hydrazinocarbonyl)-heptyl]-formamide.
N-Hydroxy-N-{(R)-2-[N′-(3-methoxy-phenyl)-hydrazinocarbonyl]-heptyl}-formamide.
N-Hydroxy-N-{(R)-2-[N′-(4-trifluoromethyl-pyrimidin-2-yl)-hydrazinocarbonyl]-heptyl}-formamide.
N-{(R)-2-[N′-(4-Cyano-phenyl)-hydrazinocarbonyl]-heptyl}-N-hydroxy-formamide.
N-{(R)-2-[N′-(2,6-Dimethyl-pyrimidin-4-yl)-hydrazinocarbonyl]-heptyl}-N-hydroxy-formamide.
N-Hydroxy-N—[(R)-2-(N′-quinoxalin-2-yl-hydrazinocarbonyl)-heptyl]-formamide.
N-Hydroxy-N-((2R)-2-{N′-(3,4-dihydro-quinoxalin-2-yl)-hydrazinocarbonyl}-heptyl)-formamide.
N-Hydroxy-N-{(R)-2-[N′-(1,3,4-trimethyl-1H-pyrazolo[3,4-b]pyridin-6-yl)-hydrazinocarbonyl]-heptyl}-formamide. 4-(N′-{(R)-2-[(Formyl-hydroxy-amino)-methyl]-heptanoyl}-hydrazino)-benzenesulfonamide.
N-Hydroxy-N-[(2R)-2-(cyclohexylmethyl)-3-oxo-3-{N′-[4-(trifluoromethyl)-pyrimidin-2-yl]-hydrazino}-propyl]-formamide.
N-Hydroxy-N-[(2R)-2-(cyclopentylmethyl)-3-oxo-3-{N′-[4-(trifluoromethyl)-pyrimidin-2-yl]-hydrazino}-propyl]-formamide.
N-{(R)-2-[N′-(Dimethyl-trifluoromethyl-pyrimidin-4-yl)-hydrazinocarbonyl]-heptyl}-N-hydroxy-formamide.
N-Hydroxy-N-{(R)-2-[N′-(6-trifluoromethyl-pyridazin-3-yl)-hydrazinocarbonyl]-heptyl}-formamide.
N-Hydroxy-N-{(R)-2-[N′-(6-trifluoromethyl-pyrimidin-4-yl)-hydrazinocarbonyl]-heptyl}-formamide.
N-Hydroxy-N-{(R)-2-[N′-(5-methyl-[1,2,4]triazolo[1,5-a]pyrimidin-7-yl)-hydrazinocarbonyl]-heptyl}-formamide.
N-Hydroxy-N-{(R)-2-[N′-(9H-purin-6-yl)-hydrazinocarbonyl]-heptyl}-formamide.
N-{(R)-2-[N′-(5-Cyano-pyrimidin-2-yl)-hydrazinocarbonyl]-heptyl}-N-hydroxy-formamide.
N-Hydroxy-N-((2R)-2-{[N′-(pyrimidin-2-yl)-hydrazino]carbonyl}-heptyl)-formamide.
N-Hydroxy-N-((2R)-2-(cyclobutylmethyl)-3-oxo-3-{N′-[4-(trifluoromethyl)-pyrimidin-2-yl]-hydrazino}-propyl)-formamide.
N-Hydroxy-N-{(R)-2-[N′-(6-imidazol-1-yl-pyrimidin-4-yl)-hydrazinocarbonyl]-heptyl}-formamide.
N—[(R)-2-(N′-Benzo[1,2,4]triazin-3-yl-hydrazinocarbonyl)-heptyl]-N-hydroxy-formamide.
N-Hydroxy-N-{(R)-2-[N′-(7-methoxy-benzo[1,2,4]triazin-3-yl)-hydrazinocarbonyl]-heptyl}-formamide.
N-Hydroxy-N-{(R)-2-[N′-(1-methyl-1H-pyrazolo[3,4-d]pyrimidin-6-yl)-hydrazinocarbonyl]-heptyl}-formamide.
N—((R)-2-{N′-[6-(5-Chloro-pyridin-3-yl-oxy)-pyridazin-3-yl]-hydrazinocarbonyl}-heptyl)-N-hydroxy-formamide.
N-Hydroxy-N-[(2R)-2-({N′-[6-(1H-pyrrol-1-yl)-3-pyridazinyl]-hydrazino}-carbonyl)-heptyl]-formamide.
N-Hydroxy-N-((2R)-2-{[N′-(9-methyl-9H-purin-6-yl)-hydrazino]-carbonyl 1-heptyl)-formamide.
N-Hydroxy-N-{(R)-2-[N-({6-morpholin-4-yl}-9H-purin-2-yl)-hydrazinocarbonyl]-heptyl}-formamide.
N-{(R)-2-[N′-(6-Fluoro-pyridin-2-yl)-hydrazinocarbonyl]-heptyl}-N-hydroxy-formamide.
N-Hydroxy-N-((2R)-2-{[N′-(1-methyl-1H-pyrazolo[3,4-d]pyrimidin-4-yl)-hydrazino]-carbonyl}-heptyl)-formamide.
N-{(R)-2-[N′-(4-Amino-6-isopropyl-[1,3,5]triazin-2-yl)-hydrazinocarbonyl]-heptyl}-N-hydroxy-formamide.
N-{(R)-2-[N′-(2,5-Dimethyl-4-nitro-2H-pyrazol-3-yl)-hydrazinocarbonyl]-heptyl}-N-hydroxy-formamide.
N-{(R)-2-[N′-(3-Chloro-1-methyl-1H-pyrazolo[3,4-d]pyrimidin-6-yl)-hydrazinocarbonyl]-heptyl}-N-hydroxy-formamide.
N-{(R)-2-[N′-(6-Dimethylamino-9H-purin-2-yl)-hydrazinocarbonyl]-heptyl}-N-hydroxy-formamide.
N-Hydroxy-N-[(2R)-4-cyclopropyl-2-({N′-[4-(trifluoromethyl)-pyrimidin-2-yl]-hydrazino}-carbonyl)-butyl]-formamide.
N-Hydroxy-N-((2R)-2-(cyclopropylmethyl)-3-oxo-3-{N′-[4-(trifluoromethyl)-pyrimidin-2-yl]-hydrazino}-propyl)-formamide.
N-Hydroxy-N-{(R)-2-[N′-methyl-N′-(4-trifluoromethyl-pyrimidin-2-yl)-hydrazinocarbonyl]-heptyl}-formamide.
2-(N′-{(R)-2-[(Formyl-hydroxy-amino)-methyl]-heptanoyl}-hydrazino)-4-trifluoromethyl-pyrimidine-5-carboxylic acid methyl ester.
2-(N′-{(R)-2-[(Formyl-hydroxy-amino)-methyl]-heptanoyl}-hydrazino)-4-trifluoromethyl-pyrimidine-5-carboxylic acid.
N-{(R)-2-[N′-(5-Fluoro-4-methoxy-pyrimidin-2-yl)-hydrazinocarbonyl]-heptyl}-N-hydroxy-formamide.
N-{(R)-2-[N′-(4-Dimethylamino-pyrimidin-2-yl)-hydrazinocarbonyl]-heptyl}-N-hydroxy-formamide.
N-Hydroxy-N-{(2R)-2-[(N′-{6-[(2-hydroxyethyl)amino]-1,3-dihydro-2H-purin-2-ylidene}-hydrazino)-carbonyl]-heptyl}-formamide.
N-{(R)-2-[N′-(5-Fluoro-4-morpholin-4-yl-pyrimidin-2-yl)-hydrazinocarbonyl]-heptyl}-N-hydroxy-formamide.
N-{(R)-2-[N′-(5-Fluoro-4-methylamino-pyrimidin-2-yl)-hydrazinocarbonyl]-heptyl}-N-hydroxy-formamide.
2-(N′-{(R)-2-[(Formyl-hydroxy-amino)-methyl]-heptanoyl}-hydrazino)-4-trifluoromethyl-pyrimidine-5-carboxylic acid dimethylamide.
N-Hydroxy-N-{(R)-2-[N′-(3-oxo-3,4-dihydro-quinoxalin-2-yl)-hydrazinocarbonyl]-heptyl}-formamide.
N-{(R)-2-Butoxy-2-[N′-(4-trifluoromethyl-pyrimidin-2-yl)-hydrazinocarbonyl]-ethyl}-N-hydroxy-formamide.
2-(N′-{(R)-2-[(Formyl-hydroxy-amino)-methyl]-heptanoyl}-hydrazino)-4-trifluoromethyl-pyrimidine-5-carboxylic acid (2-fluoro-phenyl)-amide.
2-(N′-{(R)-2-[(Formyl-hydroxy-amino)-methyl]-heptanoyl}-hydrazino)-4-trifluoromethyl-pyrimidine-5-carboxylic acid tert-butylamide.
N-Hydroxy-N—((R)-2-{N′-[(1-piperidin-1-yl-methanoyl)-trifluoromethyl-pyrimidin-2-yl]-hydrazinocarbonyl}-heptyl)-formamide.
N-{(R)-2-[N′-(5-Cyano-4-trifluoromethyl-pyrimidin-2-yl)-hydrazinocarbonyl]-heptyl}-N-hydroxy-formamide.
N-Hydroxy-N-[(2R)-2-({N′-[9-(4,4,4-trifluorobutyl)-1,9-dihydro-2H-purin-2-ylidene]-hydrazino}-carbonyl)-heptyl]-formamide.
N-Hydroxy-N—((R)-2-{N′-[(1-morpholin-4-yl-methanoyl)-trifluoromethyl-pyrimidin-2-yl]-hydrazinocarbonyl}-heptyl)-formamide.
2-(N′-{(R)-2-[(Formyl-hydroxy-amino)-methyl]-heptanoyl}-hydrazino)-4-trifluoromethyl-pyrimidine-5-carboxylic acid benzylamide.
N-Hydroxy-N-[(2R)-3-[N′-(1,2,4-benzotriazin-3-yl)-hydrazino]-2-(cyclohexylmethyl)-3-oxopropyl]-formamide.
N-Hydroxy-N-((2R)-2-(cyclohexylmethyl)-3-{N′-[7-(methyloxy)-1,2,4-benzotriazin-3-yl]-hydrazino}-3-oxopropyl)-formamide.
2-[2-((2R)-2-{[Formyl (hydroxy)amino]methyl}heptanoyl)hydrazino]-N-methyl-N-2-pyridinyl-4-(trifluoromethyl)-5-pyrimidinecarboxamide.
2-[2-((2R)-2-{[Formyl(hydroxy)amino]methyl}heptanoyl)hydrazino]-N-methyl-N-phenyl-4-(trifluoromethyl)-5-pyrimidinecarboxamide.
2-(N′-{(R)-2-[(Formyl-hydroxy-amino)-methyl]-heptanoyl}-hydrazino)-4-trifluoromethyl-pyrimidine-5-carboxylic acid morpholin-4-ylamide.
N-Hydroxy-N—((R)-2-{N′-[(N′-phenyl-hydrazinocarbonyl)-trifluoromethyl-pyrimidin-2-yl]-hydrazinocarbonyl}-heptyl)-formamide. 2-(N′-{(R)-2-[(Formyl-hydroxy-amino)-methyl]-heptanoyl}-hydrazino)-4-trifluoromethyl-pyrimidine-5-carboxylic acid piperidin-1-ylamide.
2-(N′-{(R)-2-[(Formyl-hydroxy-amino)-methyl]-heptanoyl}-hydrazino)-4-trifluoromethyl-pyrimidine-5-carboxylic acid pyrrol-1-ylamide.
N-{(R)-2-[N′-(Dimethylamino-fluoro-pyrimidin-2-yl)-hydrazinocarbonyl]-heptyl}-N-hydroxy-formamide.
N—((R)-2-{N′-[(Ethyl-methyl-amino)-fluoro-pyrimidin-2-yl]-hydrazinocarbonyl}-heptyl)-N-hydroxy-formamide.
N-Hydroxy-N-((2R)-2-(cyclopentylmethyl)-3-{N′-[7-(methyloxy)-1,2,4-benzotriazin-3-yl]-hydrazino}-3-oxopropyl)-formamide.
N-Hydroxy-N-{(R)-2-[N′-(1-methyl-1H-benzoimidazol-2-yl)-hydrazinocarbonyl]-heptyl}-formamide.
N-{(R)-2-[N′-(4-Azetidin-1-yl-5-fluoro-pyrimidin-2-yl)-hydrazinocarbonyl]-heptyl}-N-hydroxy-formamide.
N-{(R)-2-[N′-(4-Cyclopropylamino-5-fluoro-pyrimidin-2-yl)-hydrazinocarbonyl]-heptyl}-N-hydroxy-formamide.
N—[(R)-2-(N-Benzo[1,2,4]triazin-3-yl-hydrazinocarbonyl)-3-cyclopentyl-propyl]-N-hydroxy-formamide.
N-Hydroxy-N-{(R)-2-[N′-(morpholin-4-yl-trifluoromethyl-pyrimidin-2-yl)-hydrazinocarbonyl]-heptyl}-formamide.
N-Hydroxy-N—[(R)-2-(N′-{[(2-hydroxy-ethyl)-methyl-amino]-trifluoromethyl-pyrimidin-2-yl}-hydrazinocarbonyl)-heptyl]-formamide.
N-Hydroxy-N—((R)-2-{N′-[(4-methyl-piperazin-1-yl)-trifluoromethyl-pyrimidin-2-yl]-hydrazinocarbonyl}-heptyl)-formamide.
N-Hydroxy-N-((2R)-2-(cyclohexylmethyl)-3-{N′-[4-(cyclopropylamino)-5-fluoro-pyrimidin-2-yl]hydrazino}-3-oxopropyl)-formamide.
N-Hydroxy-N-((2R)-2-(cyclopentylmethyl)-3-{N′-[4-(cyclopropylamino)-5-fluoro-pyrimidin-2-yl]hydrazino}-3-oxopropyl)-formamide.
N-Hydroxy-N-[(2R)-3-{N′-[4-(azetidin-1-yl)-5-fluoro-pyrimidin-2-yl]-hydrazino}-2-(cyclopentylmethyl)-3-oxopropyl]-formamide.
N-Hydroxy-N-[(2R)-5-methyl-2-({N′-[4-(trifluoromethyl)-pyrimidin-2-yl]-hydrazino}-carbonyl)-hexyl]-formamide.
N—[(R)-2-(N′-Benzo[1,2,4]triazin-3-yl-hydrazinocarbonyl)-5-methyl-hexyl]-N-hydroxy-formamide.
N-Hydroxy-N-[(2R)-5-methyl-2-({N′-[7-(methyloxy)-1,2,4-benzotriazin-3-yl]-hydrazino}-carbonyl)-hexyl]-formamide.
N-{(R)-2-[N′-(7-Chloro-benzo[1,2,4]triazin-3-yl)-hydrazinocarbonyl]-heptyl}-N-hydroxy-formamide.
N-Hydroxy-N-((2R)-2-(cyclopentylmethyl)-3-{N′-[4-(morpholin-4-yl)-6-(trifluoromethyl)-pyrimidin-2-yl]-hydrazino}-3-oxopropyl)-formamide.
N-Hydroxy-N-((2R)-2-(cyclopentylmethyl)-3-{N′-[4-[(2-hydroxyethyl)-(methyl)-amino]-6-(trifluoromethyl)-pyrimidin-2-yl]-hydrazino}-3-oxopropyl)-formamide.
N-Hydroxy-N-[(2R)-6-methyl-2-({N′-[4-(trifluoromethyl)-pyrimidin-2-yl]-hydrazino}-carbonyl)-heptyl]-formamide.
N-Hydroxy-N-((2R)-2-{[N′-(1,2,4-benzotriazin-3-yl)-hydrazino]-carbonyl}-6-methylheptyl)-formamide.
N-Hydroxy-N-{(R)-2-[N′-(5-methyl-benzo[1,2,4]triazin-3-yl)-hydrazinocarbonyl]-heptyl}-formamide.
N—((R)-2-{N′-[(4-Ethyl-piperazin-1-yl)-trifluoromethyl-pyrimidin-2-yl]-hydrazinocarbonyl}-heptyl)-N-hydroxy-formamide.
N-Hydroxy-N-{(R)-2-[N′-(piperazin-1-yl-trifluoromethyl-pyrimidin-2-yl)-hydrazinocarbonyl]-heptyl}-formamide.
N-{(R)-2-[N′-(7-Fluoro-benzo[1,2,4]triazin-3-yl)-hydrazinocarbonyl]-heptyl}-N-hydroxy-formamide.
N-Hydroxy-N-[(2R)-2-({N′-[4-(4-ethyl-1-piperazinyl)-6-(trifluoromethyl)-pyrimidin-2-yl]-hydrazino}-carbonyl)-6-methylheptyl]-formamide.
N-Hydroxy-N-[(2R)-6-methyl-2-({N′-[4-(piperazin-1-yl)-6-(trifluoromethyl)-pyrimidin-2-yl]-hydrazino}-carbonyl)-heptyl]-formamide.
N-Hydroxy-N-[(2R)-6-methyl-2-({N′-[4-(4-methyl-piperazin-1-yl)-6-(trifluoromethyl)-pyrimidin-2-yl]-hydrazino}-carbonyl)-heptyl]-formamide.
N-Hydroxy-N-((2R)-2-{[N′-(7-chloro-1,2,4-benzotriazin-3-yl)hydrazino]carbonyl}-6-methylheptyl)-formamide.
N-Hydroxy-N-((2R)-6-methyl-2-{[N′-(5-methyl-1,2,4-benzotriazin-3-yl)-hydrazino]-carbonyl}-heptyl)-formamide.
N-Hydroxy-N-((2R)-2-{[N′-(7-fluoro-1,2,4-benzotriazin-3-yl)-hydrazino]-carbonyl}-6-methylheptyl)-formamide.
N-Hydroxy-N—((R)-2-{N′-[(2-methoxy-ethylamino)-trifluoromethyl-pyrimidin-2-yl]-hydrazinocarbonyl}-heptyl)-formamide.
N-Hydroxy-N-[(2R)-6-methyl-2-({N′-[7-(methyloxy)-1,2,4-benzotriazin-3-yl]-hydrazino}-carbonyl)-heptyl]-formamide.
N-Hydroxy-N—[(R)-2-(N′-{[4-(2-hydroxy-ethyl)-piperazin-1-yl]-trifluoromethyl-pyrimidin-2-yl}-hydrazinocarbonyl)-heptyl]-formamide.
N-Hydroxy-N—((R)-2-{N′-[(4-pyrimidin-2-yl-piperazin-1-yl)-trifluoromethyl-pyrimidin-2-yl]-hydrazinocarbonyl}-heptyl)-formamide.
N-Hydroxy-N—((R)-2-{N′-[(2-hydroxy-ethylamino)-trifluoromethyl-pyrimidin-2-yl]-hydrazinocarbonyl}-heptyl)-formamide.
N-Hydroxy-N-{(R)-2-[N′-(7-trifluoromethyl-benzo[1,2,4]triazin-3-yl)-hydrazinocarbonyl]-heptyl}-formamide.
N-Hydroxy-N-{(R)-2-[N′-(6-methyl-benzo[1,2,4]triazin-3-yl)-hydrazinocarbonyl]-heptyl}-formamide.
N-Hydroxy-N-{(2R)-2-(cyclopentylmethyl)-3-[N′-(5-methyl-1,2,4-benzotriazin-3-yl)hydrazino]-3-oxopropyl}-formramide.
N-Hydroxy-N-[(2R)-2-({N′-[4-(trifluoromethyl)-pyrimidin-2-yl]-hydrazino}-carbonyl)-octyl]-formamide.
N-Hydroxy-N-((2R)-2-{[N′-(1,2,4-benzotriazin-3-yl)hydrazino]-carbonyl}-octyl)-formamide.
N-Hydroxy-N-[(2R)-2-({N′-[7-(methyloxy)-1,2,4-benzotriazin-3-yl]-hydrazino}-carbonyl)-octyl]-formamide.
N-Hydroxy-N-{(R)-2-[N′-(7-methyl-benzo[1,2,4]triazin-3-yl)-hydrazinocarbonyl]-heptyl}-formamide.
N-{(R)-2-[N′-(6-Chloro-benzo[1,2,4]triazin-3-yl)-hydrazinocarbonyl]-heptyl}-N-hydroxy-formamide.
N-Hydroxy-N—{(R)-2-[N′-(5-methoxy-benzo[1,2,4]triazin-3-yl)-hydrazinocarbonyl]-heptyl}-formamide.
N-Hydroxy-N-{(R)-2-[N′-(1-methyl-2-oxo-1,2-dihydro-pyridin-4-yl)-hydrazinocarbonyl]-heptyl}-formamide.
N-Hydroxy-N—((R)-2-{N′-[(N′-pyridin-2-yl-hydrazino)-trifluoromethyl-pyrimidin-2-yl]-hydrazinocarbonyl}-heptyl)-formamide.
N-Hydroxy-N-{(R)-2-[N′-(4-methyl-6-morpholin-4-yl-pyrimidin-2-yl)-hydrazinocarbonyl]-heptyl}-formamide.
N—((R)-2-{N′-[4-(4-Ethyl-piperazin-1-yl)-6-methyl-pyrimidin-2-yl]-hydrazinocarbonyl}-heptyl)-N-hydroxy-formamide.
N-{(R)-2-[N′-(4,6-Dimethyl-pyrimidin-2-yl)-hydrazinocarbonyl]-heptyl}-N-hydroxy-formamide.
N-Hydroxy-N-{(R)-2-[N′-(4-trifluoromethyl-pyridin-2-yl)-hydrazinocarbonyl]-heptyl}-formamide.
N-Hydroxy-N—[(R)-2-(N′-isoquinolin-1-yl-hydrazinocarbonyl)-heptyl]-formamide.
N-Hydroxy-N—[(R)-2-(N′-quinolin-2-yl-hydrazinocarbonyl)-heptyl]-formamide.
N-{(R)-2-[N′-(1-Benzyl-2-oxo-1,2-dihydro-pyridin-4-yl)-hydrazinocarbonyl]-heptyl}-N-hydroxy-formamide.
N-Hydroxy-N-{(R)-2-[N′-(4-oxo-4H-pyrido[1,2-a][1,3,5]triazin-2-yl)-hydrazinocarbonyl]-heptyl}-formamide.
N-Hydroxy-N-{(R)-2-[N′-(4-methyl-pyrimidin-2-yl)-hydrazinocarbonyl]-heptyl}-formamide.
N-{(R)-2-[N′-(1-Butyl-2-oxo-1,2-dihydro-pyridin-4-yl)-hydrazinocarbonyl]-heptyl}-N-hydroxy-formamide.
N-Hydroxy-N-{(R)-2-[N′-(9-methyl-4-oxo-4H-pyrido[1,2-a][1,3,5]triazin-2-yl)-hydrazinocarbonyl]-heptyl}-formamide.
N-Hydroxy-N-{(R)-2-[N′-(6-oxo-4-trifluoromethyl-1,6-dihydro-pyrimidin-2-yl)-hydrazinocarbonyl]-heptyl}-formamide.
N-Hydroxy-N-{(R)-2-[N′-(methyl-trifluoromethyl-pyrimidin-2-yl)-hydrazinocarbonyl]-heptyl}-formamide.
N-Hydroxy-N-1{(R-2-[N′-(5-trifluoromethyl-pyridin-2-yl)-hydrazinocarbonyl]-heptyl}-formamide.
N-{(R)-2-[N′-(6-Ethoxy-pyridin-2-yl)-hydrazinocarbonyl]-heptyl}-N-hydroxy-formamide.
N-Hydroxy-N—[(R)-2-(N′-pyrido[2,3-e]-[1,2,4]triazin-3-yl-hydrazinocarbonyl)-heptyl]-formamide.
N—((R)-2-{N′-[1-(1-Ethyl-propyl)-2-oxo-1,2-dihydro-pyridin-4-yl]-hydrazinocarbonyl}-heptyl)-N-hydroxy-formamide.
N-Hydroxy-N—((R)-2-{N′-[2-oxo-1-(3-trifluoromethyl-benzyl)-1,2-dihydro-pyridin-4-yl]-hydrazinocarbonyl}-heptyl)-formamide.
N-Hydroxy-N—{(R)-2-[N′-(4-methyl-pyridin-2-yl)-hydrazinocarbonyl]-heptyl}-formamide.
N-Hydroxy-N-{(R)-2-[N′-(6-methoxy-pyridin-2-yl)-hydrazinocarbonyl]-heptyl}-formamide.
N-Hydroxy-N-{(R)-2-[N′-(2-oxo-1-quinolin-8-yl-methyl-1,2-dihydro-pyridin-4-yl)-hydrazinocarbonyl]-heptyl}-formamide.
N-Hydroxy-N—[(R)-2-(N′-{2-oxo-1-[2-(5,6,7,8-tetrahydro-[1,8]naphthyridin-2-yl)-ethyl]-dihydro-pyridin-4-yl}-hydrazinocarbonyl)-heptyl]-formamide.
N-{(R)-2-[N′-(4,6-Bis-ethylamino-[1,3,5]triazin-2-yl)-hydrazinocarbonyl]-heptyl}-N-hydroxy-formamide.
N-{(R)-2-[N′-(Bis-dimethylanlino-[1,3,5]triazin-2-yl)-hydrazinocarbonyl]-heptyl}-N-hydroxy-formamide.
N-{(R)-2-[N′-(4,6-Di-morpholin-4-yl-[1,3,5]triazin-2-yl)-hydrazinocarbonyl]-heptyl}-N-hydroxy-formamide.
N-Hydroxy-N—((R)-2-{N′-[4-(4-methyl-piperazin-1-yl)-6-propylamino-[1,3,5]triazin-2-yl]-hydrazinocarbonyl}-heptyl)-formamide.
N-{(R)-2-[N′-(Dimethylamino-morpholin-4-yl-[1,3,5]triazin-2-yl)-hydrazinocarbonyl]-heptyl}-N-hydroxy-formamide.
N-Hydroxy-N-{(R)-2-[N′-(6-methyl-pyridin-2-yl)-hydrazinocarbonyl]-heptyl}-formamide.
N-Hydroxy-N—((R)-2-{N′-[5-(5-phenyl-[1,3,4]oxadiazol-2-yl)-pyridin-2-yl]-hydrazinocarbonyl}-heptyl)-formamide.
N-{(R)-2-[N′-(7-tert-Butyl-1,4-dioxo-1,2,3,4-tetrahydro-pyrido[3,4-d]pyridazin-5-yl)-hydrazinocarbonyl]-heptyl}-N-hydroxy-formamide.
N—((R)-2-{N′-[4-Ethylamino-6-(4-methyl-[1,4]diazepan-1-yl)-[1,3,5]triazin-2-yl]-hydrazinocarbonyl}-heptyl)-N-hydroxy-formamide.
N—((R)-2-{N′-[4-Ethylamino-6-(4-ethyl-piperazin-1-yl)-[1,3,5]triazin-2-yl]-hydrazinocarbonyl}-heptyl)-N-hydroxy-formamide.
N-Hydroxy-N-{(R)-2-[N′-(6-trifluoromethyl-pyridin-2-yl)-hydrazinocarbonyl]-heptyl}-formamide.
N-Hydroxy-N-{(R)-2-[N′-(4-methyl-6-morpholin-4-yl-methyl-pyrimidin-2-yl)-hydrazinocarbonyl]-heptyl}-formamide.
N-Hydroxy-N-{(R)-2-[N′-(4-methyl-6-morpholin-4-yl-[1,3,5]triazin-2-yl)-hydrazinocarbonyl]-heptyl}-formamide.
N-Hydroxy-N-{(2R)-2-(cyclopentylmethyl)-3-[N′-(4,6-dimethyl-2-pyrimidinyl)-hydrazino]-3-oxopropyl}-formamide.
N-Hydroxy-N-{(R)-2-[N′-(4-methyl-6-pyrrolidin-1-yl-methyl-pyrimidin-2-yl)-hydrazinocarbonyl]-heptyl}-formamide.
N-{(R)-2-[N′-(4-Dimethylaminomethyl-6-methyl-pyrimidin-2-yl)-hydrazinocarbonyl]-heptyl}-N-hydroxy-formamide.
N-Hydroxy-N—((R)-2-{N′-[4-methyl-6-(4-methyl-piperazin-1-yl-methyl)-pyrimidin-2-yl]-hydrazinocarbonyl}-heptyl)-formamide.
N-Hydroxy-N-{(R)-2-[N′-(5-methyl-pyridin-2-yl)-hydrazinocarbonyl]-heptyl}-formamide.
N—((R)-2-{N′-[Dimethylamino-(4-methyl-[1,4]diazepan-1-yl)-[1,3,5]triazin-2-yl]-hydrazinocarbonyl}-heptyl)-N-hydroxy-formamide.
N-Hydroxy-N-{(R)-2-[N′-(4-methyl-6-pyrrolidin-1-yl-[1,3,5]triazin-2-yl)-hydrazinocarbonyl]-heptyl}-formamide.
N-Hydroxy-N—((R)-2-{N′-[4-methyl-6-)-4-pyrrolidin-1-yl-piperidin-1-yl)-[1,3,5]triazin-2-yl]-hydrazinocarbonyl}-heptyl)-formamide.
N—((R)-2-{N′[(Ethyl-methyl-amino)-methyl-[1,3,5]triazin-2-yl]-hydrazinocarbonyl}-heptyl)-N-hydroxy-formamide.
N—((R)-2-{N′-[(4-(4-Ethyl-piperazin-1-yl)-6-methyl-[1,3,5]triazin-2-yl]-hydrazinocarbonyl}-heptyl)-N-hydroxy-formamide.
N-Hydroxy-N-[(2R)-7,7,7-trifluoro-2-({N′-[4-(trifluoromethyl)-pyrimidin-2-yl]-hydrazino}-carbonyl)-heptyl]-formamide.
N-Hydroxy-N-((2R)-7,7,7-trifluoro-2-{[N′-(5-methyl-1,2,4-benzotriazin-3-yl)-hydrazino]-carbonyl}-heptyl)-formamide.
N-Hydroxy-N-((2R)-7,7,7-trifluoro-2-{[N′-(7-methyl-1,2,4-benzotriazin-3-yl)-hydrazino]-carbonyl}-heptyl)-formamide.
N-Hydroxy-N-{(R)-2-[N′-(4-methylamino-6-morpholin-4-yl-[1,3,5]-2-yl)-hydrazinocarbonyl]-heptyl}-formamide.
N—((R)-2-{N′-[4-(4-Ethyl-piperazin-1-yl)-6-methylamino-[1,3,5]triazin-2-yl]-hydrazinocarbonyl}-heptyl)-N-hydroxy-formamide.
N—{(R)-2-[N′-(4-Ethylamino-6-morpholin-4-yl-[1,3,5]triazin-2-yl)-hydrazinocarbonyl]-heptyl}-N-hydroxy-formamide.
N-Hydroxy-N-{(R)-2-[N′-(4,6,7-trimethyl-7,8-dihydro-pterin-2-yl)-hydrazinocarbonyl]-heptyl}-formamide.
N-Hydroxy-N-{(R)-2-[N′-(4,6,7-trimethyl-pteridin-2-yl)-hydrazinocarbonyl]-heptyl}-formamide.
N-Hydroxy-N-{(R)-2-[N′-(methoxymethoxymethyl-trifluoromethyl-pyrimidin-2-yl)-hydrazinocarbonyl]-heptyl}-formamide.
N-Hydroxy-N—((R)-2-{N′-[4-methyl-6-(1-piperidin-1-yl-methanoyl)-pyrimidin-2-yl]-hydrazinocarbonyl}-heptyl)-formamide.
2-(N′-{(R)-2-[(Formyl-hydroxy-amino)-methyl]-heptanoyl}-hydrazino)-6-methyl-pyrimidine-4-carboxylic acid cyclopropylamide.
2-(N′-{(R)-2-[(Formyl-hydroxy-amino)-methyl]-heptanoyl}-hydrazino)-6-methyl-pyrimidine-4-carboxylic acid diisopropylamide.
N-{(R)-2-[N′-(5-Cyano-pyridin-2-yl)-hydrazinocarbonyl]-heptyl}-N-hydroxy-formamide.
N-{(R)-2-[N′-(4,6-Diethyl-[1,3,5]triazin-2-yl)-hydrazinocarbonyl]-heptyl}-N-hydroxy-formamide.
N-{(R)-4-Cyclopentyl-2-[N′-(4-trifluoromethyl-pyrimidin-2-yl)-hydrazinocarbonyl]-butyl}-N-hydroxy-formamide.
N-{(R)-4-Cyclopentyl-2-[N′-(7-methyl-benzo[1,2,4]triazin-3-yl)-hydrazinocarbonyl]-butyl}-N-hydroxy-formamide.
N-{(R)-4-Cyclopentyl-2-[N′-(5-methyl-benzo[1,2,4]triazin-3-yl)-hydrazinocarbonyl]-butyl}-N-hydroxy-formamide.
N-Hydroxy-N—((R)-2-{N′-[6-(4-methyl-piperazin-1-yl-methyl)-pyridin-2-yl]-hydrazinocarbonyl}-heptyl)-formamide.
N—((R)-2-{N′-[5-(4,6-Dimethoxy-pyrimidin-2-yl)-pyridin-2-yl]-hydrazinocarbonyl}-heptyl)-N-hydroxy-formamide.
N-{(R)-2-[N′-(Diethylamino-methyl-[1,3,5]triazin-2-yl)-hydrazinocarbonyl]-heptyl}-N-hydroxy-formamide.
N-Hydroxy-N—[(R)-2-(N′-{[(2-methoxy-ethyl)-methyl-amino]-methyl-[1,3,5]triazin-2-yl}-hydrazinocarbonyl)-heptyl]-formamide.
N—((R)-1-{N′-[4-(2,6-Dimethyl-morpholin-4-yl)-6-methyl-[1,3,5]triazin-2-yl]-hydrazinocarbonyl}-heptyl)-N-hydroxy-formamide.
N-{(R)-2-[N′-(5-Fluoro-4-methyl-6-morpholin-4-yl-pyrimidin-2-yl)-hydrazinocarbonyl]-heptyl}-N-hydroxy-formamide.
N-{(R)-2-[N′-(4-Ethyl-6-morpholin-4-yl-[1,3,5]triazin-2-yl)-hydrazinocarbonyl]-heptyl}-N-hydroxy-formamide.
N-{(R)-2-[N′-(Ethyl-methyl-amino)-[1,3,5]triazin-2-yl)-hydrazinocarbonyl]-heptyl}-N-hydroxy-formamide.
N—((R)-2-{N′-[4-Ethyl-6-(4-ethyl-piperazin-1-yl)-[1,3,5]triazin-2-yl]-hydrazinocarbonyl}-heptyl)-N-hydroxy-formamide.
N—((R)-2-{N′-[5-Fluoro-4-methyl-6-(4-methyl-piperazin-1-yl)-pyrimidin-2-yl]-hydrazinocarbonyl}-heptyl)-N-hydroxy-formamide.
N-{(R)-2-[N′-(Dimethylamino-ethyl-[1,3,5]triazin-2-yl)-hydrazinocarbonyl]-heptyl}-N-hydroxy-formamide.
N—((R)-2-{N′-[5-Fluoro-4-methyl-6-(4-methyl-[1,4]diazepan-1-yl)-pyrimidin-2-yl]-hydrazinocarbonyl}-heptyl)-N-hydroxy-formamide.
N-{(R)-4-Cyclopentyl-2-[N′-(morpholin-4-yl-trifluoromethyl-pyrimidin-2-yl)-hydrazinocarbonyl]-butyl}-N-hydroxy-formamide.
N—[(R)-2-(N′-{Ethyl-[(2-methoxy-ethyl)-methyl-amino]-[1,3,5]triazin-2-yl}-hydrazinocarbonyl)-heptyl]-N-hydroxy-formamide.
N-{(R)-2-[N′-(Dimethylamino-methyl-pyrimidin-2-yl)-hydrazinocarbonyl]-heptyl}-N-hydroxy-formamide.
N-{(R)-2-[N′-(4-Cyclopropylamino-6-methyl-pyrimidin-2-yl)-hydrazinocarbonyl]-heptyl}-N-hydroxy-formamide.
N-{(R)-2-Cyclohexyl-2-[N′-(4-trifluoromethyl-pyrimidin-2-yl)-hydrazinocarbonyl]-ethyl}-N-hydroxy-formamide.
N-{(R)-2-Cyclohexyl-2-[N′-(7-methoxy-benzo[1,2,4]triazin-3-yl)-hydrazinocarbonyl]-ethyl}-N-hydroxy-formamide.
N-{(R)-2-Cyclohexyl-2-[N′-(5-methyl-benzo[1,2,4]triazin-3-yl)-hydrazinocarbonyl]-ethyl}-N-hydroxy-formamide.
N-{(R)-4,4-Dimethyl-2-[N′-(4-trifluoromethyl-pyrimidin-2-yl)-hydrazinocarbonyl]-pentyl}-N-hydroxy-formamide.
N-{(R)-4,4-Dimethyl-2-[N′-(7-methyl-benzo[1,2,4]triazin-3-yl)-hydrazinocarbonyl]-pentyl}-N-hydroxy-formamide.
N-{(R)-4,4-Dimethyl-2-[N′-(5-methyl-benzo[1,2,4]triazin-3-yl)-hydrazinocarbonyl]-pentyl}-N-hydroxy-formamide.
N-{(R)-4,4-Dimethyl-2-[N′-(morpholin-4-yl-trifluoromethyl-pyrimidin-2-yl)-hydrazinocarbonyl]-pentyl}-N-hydroxy-formamide.
N—((R)-2-{N′-[Ethyl-(methyl-pyridin-2-yl-amino)-[1,3,5]triazin-2-yl]-hydrazinocarbonyl}-heptyl)-N-hydroxy-formamide.
N-{(R)-2-[N′-(4-Cyclopropylamino-6-ethyl-[1,3,5]triazin-2-yl)-hydrazinocarbonyl]-heptyl}-N-hydroxy-formamide.
N-Hydroxy-N—[(R)-2-[N′-(5-methyl-benzo[1,2,4]triazin-3-yl)-hydrazinocarbonyl]-3-(1-methyl-cyclopentyl)-propyl]-formamide.
N-Hydroxy-N—[(R)-2-[N′-(7-methoxy-benzo[1,2,4]triazin-3-yl)-hydrazinocarbonyl]-3-(1-methyl-cyclopentyl)-propyl]-formamide.
N-Hydroxy-N-{(R)-3-(1-methyl-cyclopentyl)-2-[N′-(4-trifluoromethyl-pyrimidin-2-yl)-hydrazinocarbonyl]-propyl}-formamide.
N-Hydroxy-N-{(R)-2-[N′-(4-isopropyl-6-morpholin-4-yl-[1,3,5]triazin-2-yl)-hydrazinocarbonyl]-heptyl}-formamide.
N-Hydroxy-N-{(2R)-2-(cyclopentylmethyl)-3-[N′-(4-methyl-2-pyrimidinyl)-hydrazino]-3-oxopropyl}-formamide.
N-Hydroxy-N-[(2R)-6,6,6-trifluoro-2-({N′-[4-(trifluoromethyl)-pyrimidin-2-yl]-hydrazino}-carbonyl)-hexyl]-formamide.
N-{(R)-2-[N′-(5,7-Dimethyl-benzo[1,2,4]triazin-3-yl)-hydrazinocarbonyl]-heptyl}N-hydroxy-formamide.
N-{(R)-2-[N′-(3,6-Dimethyl-pyrazin-2-yl)-hydrazinocarbonyl]-heptyl}-N-hydroxy-formamide.
N—((R)-2-{N′-[4-(4-Ethyl-piperazine-1-yl)-6-isopropyl-[1,3,5]triazin-2-yl]-hydrazinocarbonyl}-heptyl)-N-hydroxy-formamide.
N-{(R)-2-[N′-(4-Dimethylamino-6-isopropyl-[1,3,5]triazin-2-yl)-hydrazinocarbonyl]-heptyl}-N-hydroxy-formamide.
N-Hydroxy-N-{(R)-2-[N′-(methyl-trifluoromethyl-pyridin-2-yl)-hydrazinocarbonyl]-heptyl}-formamide.
2-(N′-{(R)-2-[(Formyl-hydroxy-amino)-methyl]-heptanoyl}-hydrazino)-6,N,N-trimethyl-isonicotinamide.
N-Hydroxy-N-[(2R)-2-({N′-[3-amino-6-(trifluoromethyl)-pyridin-2-yl]-hydrazino}-carbonyl)-heptyl]-formamide.
N-Hydroxy-N—[(R)-2-(N′-{4-isopropyl-6-[(2-methoxy-ethyl)-methyl-amino]-[1,3,5]triazin-2-yl}-hydrazinocarbonyl)-heptyl]-formamide.
N-{(R)-3-Cyclopentyl-2-[N′-(4-ethyl-6-morpholin-4-yl-[1,3,5]triazin-2-yl)-hydrazinocarbonyl]-propyl}-N-hydroxy-formamide.
N-Hydroxy-N-{(R)-2-[N′-(4-morpholin-4-yl-6-propyl-[1,3,5]triazin-2-yl)-hydrazinocarbonyl]-heptyl}-formamide.
N—((R)-2-{N′-[4-(4-Ethyl-piperazin-1-yl)-6-propyl-[1,3,5]triazin-2-yl]-hydrazinocarbonyl}-heptyl)-N-hydroxy-formamide.
N-{(R)-5,5-Dimethyl-2-[N′(4-trifluoromethyl-pyrimidin-2-yl)-hydrazinocarbonyl]-hexyl}-N-hydroxy-formamide.
N-{(R)-5,5-Dimethyl-2-[N′-(7-methyl-benzo[1,2,4]triazin-3-yl)-hydrazinocarbonyl]-hexyl}-N-hydroxy-formamide.
N-{(R)-5,5-Dimethyl-2-[N′-(5-methyl-benzo[1,2,4]triazin-3-yl)-hydrazinocarbonyl]-hexyl}-N-hydroxy-formamide.
N-{(R)-5,5-Dimethyl-2-[N′-(morpholin-4-yl-trifluoromethyl-pyrimidin-2-yl)-hydrazinocarbonyl]-hexyl}-N-hydroxy-formamide.
N-{(R)-4-Ethyl-2-[N′-(4-trifluoromethyl-pyrimidin-2-yl)-hydrazino carbonyl]-hexyl}-N-hydroxy-formamide.
N-{(R)-4-Ethyl-2-[N′-(7-methyl-benzo[1,2,4]triazin-3-yl)-hydrazinocarbonyl]-hexyl}-N-hydroxy-formamide.
N-{(R)-4-Ethyl-2-[N′-(morpholin-4-yl-trifluoromethyl-pyrimidin-2-yl)-hydrazinocarbonyl]-hexyl}-N-hydroxy-formamide.
N—((R)-3-Cyclopentyl-2-{N′-[4-ethyl-6-(4-ethyl-piperazin-1-yl)-[1,3,5]triazin-2-yl]-hydrazinocarbonyl}-propyl)-N-hydroxy-formamide.
N-{(R)-3-Cyclopentyl-2-[N′-(4-cyclopropylamino-6-ethyl-[1,3,5]triazin-2-yl)-hydrazinocarbonyl]-propyl}-N-hydroxy-formamide.
N-{(R)-4-Ethyl-2-[N′-(5-methyl-benzo[1,2,4]triazin-3-yl)-hydrazinocarbonyl]-hexyl}-N-hydroxy-formamide.
N-Hydroxy-N—[(R)-2-(N′-{[(2-methoxy-ethyl)-methyl-amino]-propyl-[1,3,5]triazin-2-yl}-hydrazinocarbonyl)-heptyl]-formamide.
N-{(R)-2-[N′-(Dimethylamino-propyl-[1,3,5]triazin-2-yl)-hydrazinocarbonyl]-heptyl}-N-hydroxy-formamide.
N-{(R)-2-[N′-(4-Ethyl-pyrimidin-2-yl)-hydrazinocarbonyl]-heptyl}-N-hydroxy-formamide.
N-Hydroxy-N-{(R)-2-[N′-(4-isopropyl-pyrimidin-2-yl)-hydrazinocarbonyl]-heptyl}-formamide.
N-{(R)-2-[N′-(4-Cyclopropyl-6-morpholin-4-yl-[1,3,5]triazin-2-yl)-hydrazinocarbonyl]-heptyl}-N-hydroxy-formamide.
N-Hydroxy-N-[(2R)-2-({N′-[4-(pyridin-2-yl)-pyrimidin-2-yl]-hydrazino}-carbonyl)-heptyl]-formamide.
N—((R)-2-{N′-[4-Cyclopropyl-6-(4-ethyl-piperazin-1-yl)-[1,3,5]triazin-2-yl]-hydrazinocarbonyl}-heptyl)-N-hydroxy-formamide.
N-{(R)-2-[N′-(Cyclopropyl-dimethylamino-[1,3,5]triazin-2-yl)-hydrazinocarbonyl]-heptyl}-N-hydroxy-formamide.
N—((R)-2-{N′-[Cyclopropyl-(ethyl-methyl-amino)-[1,3,5]triazin-2-yl]-hydrazinocarbonyl}-heptyl)-N-hydroxy-formamide.
N-{(R)-2-[N′-(4-Cyclopropyl-6-pyrrolidin-1-yl[1,3,5]triazin-2-yl)-hydrazinocarbonyl]-heptyl}-N-hydroxy-formamide.
N-{(R)-2-[N′ (4,6-Dicyclopropyl-[1,3,5]triazin-2-yl)-hydrazinocarbonyl]-heptyl}-N-hydroxy-formamide.
N-Hydroxy-N—[(R)-2-[N′-(5-methyl-benzo[1,2,4]triazin-3-yl)-hydrazinocarbonyl]-3-(2-methyl-cyclopentyl)-propyl]-formamide.
N—[(R)-2′ [N′-(Dimethylamino-ethyl-[1,3,5]triazin-2-yl)-hydrazinocarbonyl]-3-(2-methyl-cyclopentyl)-propyl]-N-hydroxy-formamide.
N-Hydroxy-N—[(R)-2-[N′-(4-trifluoromethyl-pyrimidin-2-yl)-hydrazinocarbonyl]-3-(2-methyl-cyclopentyl)-propyl]-formamide.
N-{(R)-2-[N′-(5-Ethyl-pyrimidin-2-yl)-hydrazinocarbonyl]-heptyl}-N-hydroxy-formamide.
N-Hydroxy-N-{(2R)-2-(cyclopentylmethyl)-3-[N′-(7-methyl-1,2,4-benzotriazin-3-yl)-hydrazino]-3-oxopropyl}-formamide.
N-Hydroxy-N-[(2R)-2-(cyclopentylmethyl)-3-(N′-{4-ethyl-6-[ethyl(methyl)amino]-1,3,5-triazin-2-yl}-hydrazino)-3-oxopropyl]-formamide.
N-Hydroxy-N-((2R)-2-(cyclopentylmethyl)-3-{N′-[4-(dimethylamino)-6-ethyl-1,3,5-triazin-2-yl]-hydrazino}-3-oxopropyl)-formamide.
N—((R)-2-{N′-[4-Ethyl-6-(4-isopropyl-piperazin-1-yl)-[1,3,5]triazin-2-yl]-hydrazinocarbonyl}-heptyl)-N-hydroxy-formamide.
N-Hydroxy-N-[(2R)-3-[N′-(6-chloro-1,2,4-benzotriazin-3-yl)-hydrazino]-2-(cyclopentylmethyl)-3-oxopropyl]-formamide.
N-{(R)-4,4-Dimethyl-2-[N′-(4-trifluoromethyl-pyrimidin-2-yl)-hydrazinocarbonyl]-hexyl}-N-hydroxy-formamide.
N-{(R)-4,4-Dimethyl-2-[N′-(7-methyl-benzo[1,2,4]triazin-3-yl)-hydrazinocarbonyl]-hexyl}-N-hydroxy-formamide.
N-{(R)-4,4-Dimethyl-2-[N′-(5-methyl-benzo[1,2,4]triazin-3-yl)-hydrazinocarbonyl]-hexyl}-N-hydroxy-formamide.
N-{(R)-4,4-Dimethyl-2-[N′-(morpholin-4-yl-trifluoromethyl-pyrimidin-2-yl)-hydrazinocarbonyl]-hexyl}-N-hydroxy-formamide.
N-Hydroxy-N-{(R)-2-[N′-(5-phenyl-[1,2,4]triazin-3-yl)-hydrazinocarbonyl]-heptyl}-formamide.
N-{(R)-2-[N′-(4-Ethyl-6-morpholin-4-yl-pyrimidin-2-yl)-hydrazinocarbonyl]-heptyl}-N-hydroxy-formamide.
N—((R)-2-{N′-[4-Ethyl-6-(4-methyl-piperazin-1-yl)-pyrimidin-2-yl]-hydrazinocarbonyl}-heptyl)-N-hydroxy-formamide.
N-{(R)-2-[N′-(5-Ethyl-4-methyl-pyrimidin-2-yl)-hydrazinocarbonyl]-heptyl}-N-hydroxy-formamide.
N—((R)-2-{N′-[4-Ethyl-6-(4-propyl-piperazin-1-yl)-[1,3,5]triazin-2-yl]-hydrazinocarbonyl}-heptyl)-N-hydroxy-formamide.
N-Hydroxy-N—((R)-2-{N′-[6-(4-pyrimidin-2-yl-piperazin-1-yl-methyl)-pyridin-2-yl]-hydrazinocarbonyl}-heptyl)-formamide.
N-Hydroxy-N—((R)-2-{N′-[6-(3-[1,2,4]triazol-1-yl-methyl-[1,2,4]triazol-1-yl)-pyridin-2-yl]-hydrazinocarbonyl}-heptyl)-formamide.
N-{(R)-3-Bicyclo[2.2.1]hept-7-yl-2-[N′-(4-trifluoromethyl-pyrimidin-2-yl)-hydrazinocarbonyl]-propyl}-N-hydroxy-formamide.
N-{(R)-3-Bicyclo[2.2.1]hept-7-yl-2-[N′-(morpholin-4-yl-trifluoromethyl-pyrimidin-2-yl)-hydrazinocarbonyl]-propyl}-N-hydroxy-formamide.
N-hydroxy-N—[(R)-2-(N′-pyridin-3-yl-hydrazinocarbonyl)-heptyl]-formamide. 4-{4-Ethyl-6-[2-((2R)-2-{[formyl(hydroxy)amino]-methyl}-heptanoyl)-hydrazino]-1,3,5-triazin-2-yl}-1-methyl-1-propylpiperazin-1-ium iodide.
N-{(R)-3-Bicyclo[2.2.1]hept-7-yl-2-[N′-(5-methyl-benzo[1,2,4]triazin-3-yl)-hydrazinocarbonyl]-propyl}-N-hydroxy-formamide.
N-{(R)-2-[N′-(4-Azetidin-1-yl-6-ethyl-[1,3,5]triazin-2-yl)-hydrazinocarbonyl]-heptyl}-N-hydroxy-formamide.
N-{(R)-2-Cyclopentyl-2-[N′-(4-trifluoromethyl-pyrimidin-2-yl)-hydrazinocarbonyl]-ethyl}-N-hydroxy-formamide.
N—{(R)-2-Cyclopentyl-2-[N′-(morpholin-4-yl-4-trifluoromethyl-pyrimidin-2-yl)-hydrazinocarbonyl]-ethyl}-N-hydroxy-formamide.
N-{(R)-2-Cyclopentyl-2-[N′-(4-methyl-pyridin-2-yl)-hydrazinocarbonyl]-ethyl}-N-hydroxy-formamide.
N-{(R)-2-Cyclopentyl-2-[N′-(dimethylamino-ethyl-[1,3,5]triazin-2-yl)-hydrazinocarbonyl]-ethyl}-N-hydroxy-formamide.
N-{(R)-2-Cyclopentyl-2-[N′-(7-methyl-benzo[1,2,4]triazin-3-yl)-hydrazinocarbonyl]-ethyl}-N-hydroxy-formamide.
N-{(R)-2-Cyclopentyl-2-[N′-(5-methyl-benzo[1,2,4]triazin-3-yl)-hydrazinocarbonyl]-ethyl}-N-hydroxy-formamide.
N-Hydroxy-N-{(R)-2-[N′-(5-methyl-benzo[1,2,4]triazin-3-yl)-hydrazinocarbonyl]-2-(4-methyl-cyclohexyl)-ethyl}-formamide.
N-Hydroxy-N—(R)-2-[N′-(7-methyl-benzo[{,2,4]triazin-3-yl)-hydrazinocarbonyl]-2-(4-methyl-cyclohexyl)-ethyl}-formamide.
N-Hydroxy-N-{(R)-2-(4-methyl-cyclohexyl)-2-[N′-(4-trifluoromethyl-pyrimidin-2-yl)-hydrazinocarbonyl]-ethyl}-formamide.
N-Hydroxy-N-{(R)-2-(4-methyl-cyclohexyl)-2-[N′-(morpholin-4-yl-trifluoromethyl-pyrimidin-2-yl)-hydrazinocarbonyl]-ethyl}-formamide.
N-Hydroxy-N-{(R)-2-(4-methyl-cyclohexyl)-2-[N′-(4-methyl-pyridin-2-yl)-hydrazinocarbonyl]-ethyl}-formamide.
N-{(R)-2-[N′-(Dimethylamino-ethyl-[1,3,5]triazin-2-yl)-hydrazinocarbonyl]-2-(4-methyl-cyclohexyl)-ethyl}-N-Hydroxy-formamide.
N-{(R)-2-[N′-(6,7-Dihydro-5H-cyclopentapyrimidin-2-yl)-hydrazinocarbonyl]-heptyl}-N-hydroxy-formamide.
N—((R)-2-[N′-[4-Ethyl-6-((S)-2-hydroxymethyl-pyrrolidin-1-yl)-[1,3,5]triazin-2-yl]-hydrazinocarbonyl]-heptyl)-N-hydroxy-formamide.
N-{(R)-2-[N′-(Dimethylamino-pyridin-3-yl-pyrimidin-2-yl)-hydrazinocarbonyl]-heptyl}-N-hydroxy-formamide.
N-{(R)-2-[N′-(Dimethylamino-pyridin-4-yl-pyrimidin-2-yl)-hydrazinocarbonyl]-heptyl}-N-hydroxy-formamide.
N-Hydroxy-N-{(R)-2-N′-(5,6,7,8-tetrahydro-benzo[1,2,4]triazin-3-yl)-hydrazinocarbonyl]-heptyl}-formamide.
N-{(R)-2-[N′-(5,6-Diethyl-[1,2,4]triazin-3-yl)-hydrazinocarbonyl]-heptyl}-N-hydroxy-formamide.
N-Hydroxy-N-{(R)-2-[N′-[5-(4-hydroxy-phenyl)-[1,2,4]triazin-3-yl]-hydrazinocarbonyl}-heptyl)-formamide.
N—[(R)-2-(N′-{[(2-Dimethylamino-ethyl)-methyl-amino]-ethyl-[1,3,5]triazin-2-yl}-hydrazinocarbonyl)-heptyl]-N-hydroxy-formamide.
N-{(R)-2-[N′-(2-Dimethylamino-quinazolin-4-yl)-hydrazinocarbonyl]-heptyl}-N-hydroxy-formamide.
N-Hydroxy-N-{(R)-2-[N′-(3-methanesulfonyl-4,6-dimethyl-pyridin-2-yl)-hydrazinocarbonyl]-heptyl}-formamide.
N—((R)-2-{N′-[4-Ethyl-6-(3-hydroxy-piperidin-1-yl)-[1,3,5]triazin-2-yl]-hydrazinocarbonyl}-heptyl)-N-hydroxy-formamide.
N—[(R)-2-(N′-[4,5′]Bipyrinidinyl-2-yl-hydrazinocarbonyl)-heptyl]-N-hydroxy-formamide.
N—((R)-2-{N′-[(Cyclopropyl-methyl-amino)-ethyl-[1,3,5]triazin-2-yl]-hydrazinocarbonyl}-heptyl)-N-hydroxy-formamide.
N—((R)-2-{N′-[4-Ethyl-6-((R)-3-hydroxy-pyrrolidin-1-yl)-[1,3,5]triazin-2-yl]-hydrazinocarbonyl}-heptyl)-N-hydroxy-formamide.
N-Hydroxy-N—[(R)-2-(N′-[3,3′]Bipyridinyl-5-yl-hydrazinocarbonyl)-heptyl]-formamide.
N-Hydroxy-N—[(R)-2-(N′-(5-morpholin-4-yl-pyridin-3-yl)-hydrazinocarbonyl)-heptyl]-formamide.
N-Hydroxy-N-{(R)-2-[N′-(4-pyridin-3-yl-pyrimidin-2-yl)-hydrazinocarbonyl]-heptyl}-formamide.
N-Hydroxy-N-{(R)-2-[N′-(5,6,7,8-tetrahydro-quinazolin-2-yl)-hydrazinocarbonyl]-heptyl}-formamide.
N—[(R)-2-(N′-{[Cyclopropyl-1-(1-methyl-piperidin-4-yl)-amino]-ethyl-[1,3,5]triazin-2-yl}-hydrazinocarbonyl)-heptyl]-N-hydroxy-formamide.
N—((R)-2-{N′-[4-((R)-3-Dimethylamino-pyrrolidin-1-yl)-6-ethyl-[1,3,5]triazin-2-yl]-hydrazinocarbonyl}-heptyl)-N-hydroxy-formamide.
N-Hydroxy-N—[(R)-2-(N′-[5-(1H-pyrrol-2-yl)-pyridin-3-yl]-hydrazinocarbonyl)-heptyl]-formamide.
N-Hydroxy-N—[(R)-2-(N′-[(4-methyl-piperazin-1-yl)-trifluoromethyl-pyrimidin-4-yl]-hydrazinocarbonyl)-heptyl]-formamide.
N-Hydroxy-N—[(R)-2-(N′-(5-Furan-3-yl-pyridin-3-yl)-hydrazinocarbonyl)-heptyl]-formamide.
N-{(R)-5,5-Dimethyl-2-[N′-(4-trifluoromethyl-pyrimidin-2-yl)-hydrazinocarbonyl]-heptyl}-N-hydroxy-formamide.
N-{(R)-5,5-Dimethyl-2-[N′-(7-methyl-benzo[1,2,4]triazin-3-yl)-hydrazinocarbonyl]-heptyl}-N-hydroxy-formamide.
N-{(R)-5,5-Dimethyl-2-(4-methyl-pyridin-2-yl)-hydrazinocarbonyl]-heptyl}-N-hydroxy-formamide.
N-{(R)-2-Cycloheptyl-2-[N′-(4-trifluoromethyl-pyrimidin-2-yl)-hydrazinocarbonyl]-ethyl}-N-hydroxy-formamide.
N-{(R)-2-Cycloheptyl-2-[N′-(4-methyl-pyridin-2-yl)-hydrazinocarbonyl]-ethyl}-N-hydroxy-formamide.
N-{(R)-2-Cycloheptyl-2-[N′-(dimethylamino-ethyl-[1,3,5]triazin-2-yl)-hydrazinocarbonyl]-ethyl}-N-hydroxy-formamide.
N-{(R)-2-Cycloheptyl-2-[N′-(7-methyl-benzo[1,2,4]triazin-3-yl)-hydrazinocarbonyl]-ethyl}-N-hydroxy-formamide.
N—((R)-2-{N′-[4-Ethyl-6-(4-hydroxy-piperidin-1-yl)-[1,3,5]triazin-2-yl]-hydrazinocarbonyl}-heptyl)-N-hydroxy-formamide.
N-{(R)-5,5-Dimethyl-2-[N′-(5-methyl-benzo[1,2,4]triazin-3-yl)-hydrazinocarbonyl]-heptyl}-N-hydroxy-formamide.
N-{(R)-2-[N′-(4-Dimethylamino-quinazolin-2-yl)-hydrazinocarbonyl]-heptyl}-N-hydroxy-formamide.
N-Hydroxy-N-{(R)-2-[N′-(4-pyridin-4-yl-pyrimidin-2-yl)-hydrazinocarbonyl]-heptyl}-formamide.
N-Hydroxy-N—((R)-2-{N′-[4-(3-hydroxymethyl-phenyl)-pyrimidin-2-yl]-hydrazinocarbonyl}-heptyl)-formamide.
N-Hydroxy-N—((R)-2-{N′-[4-(4-hydroxymethyl-phenyl)-pyrimidin-2-yl]-hydrazinocarbonyl}-heptyl)-formamide.
N—((R)-2-{N′-[4-Ethyl-6-(3-methoxy-piperidin-1-yl)-[1,3,5]triazin-2-yl]-hydrazinocarbonyl}-heptyl)-N-hydroxy-formamide.
N-Hydroxy-N-{(R)-2-[N′-(7-methyl-benzo[1,2,4]triazin-3-yl)-hydrazinocarbonyl]-2-(4-methyl-cyclohexyl)-ethyl}-formamide.
N—[(R)-2-{N′-[Ethyl-(ethyl-methylamino)-[1,3,5]triazin-2-yl]-hydrazinocarbonyl}-2-(4-methyl-cyclohexyl)-ethyl}-N-hydroxy-formamide.
N—[(R)-2-{N′-[Ethyl-(ethyl-methylamino)-[1,3,5]triazin-2-yl]-hydrazinocarbonyl}-2-(4-methyl-cyclohexyl)-ethyl}-N-hydroxy-formamide.
N-Hydroxy-N—{(R)-2-[N′-(7-methyl-benzo[1,2,4]triazin-3-yl)-hydrazinocarbonyl]-2-(4-methyl-cyclohexyl)-ethyl}-formamide.
N—((R)-2-{N′-[4-(2,6-Dimethoxy-phenyl)-pyrimidin-2-yl]-hydrazinocarbonyl}-heptyl)-N-hydroxy-formamide.
N—((R)-2-{N′-[4-Ethyl-6-((R)-3-methoxy-pyrrolidin-1-yl)-[1,3,5]triazin-2-yl]-hydrazinocarbonyl}-heptyl)-N-hydroxy-formamide.
N—((R)-2-{N′-[4-Ethyl-6-(4-methoxy-piperidin-1-yl)-[1,3,5]triazin-2-yl]-hydrazinocarbonyl}-heptyl)-N-hydroxy-formamide.
N-Hydroxy-N—[(R)-2-(N′-(6-pyrrolidin-1-yl-pyrimidin-4-yl)-hydrazinocarbonyl)-heptyl]-formamide.
N-Hydroxy-N—[(R)-2-(N′-[6-(4-methyl-piperazin-1-yl)-pyrimidin-4-yl])-hydrazinocarbonyl)-heptyl]-formamide.
N-{(R)-2-[N′-(6-Dimethylamino-pyrimidin-4-yl)-hydrazinocarbonyl]-heptyl}-N-hydroxy-formamide.
N-{(R)-2-[N′-(Pyridin-4-yl-trifluoromethyl-pyrimidin-4-yl)-hydrazinocarbonyl]-heptyl}-N-hydroxy-formamide.
N-{(R)-2-[N′-(Pyridin-3-yl-trifluoromethyl-pyrimidin-4-yl)-hydrazinocarbonyl]-heptyl}-N-hydroxy-formamide.
N-{(R)-2-[N′-(2-Ethylamino-6-trifluoromethyl-pyrimidin-4-yl)-hydrazinocarbonyl]-heptyl}-N-hydroxy-formamide.
N-Hydroxy-N—((R)-2-{N′-[5-(4-methoxy-phenyl)-[1,2,4]triazin-3-yl]-hydrazinocarbonyl}-heptyl)-formamide.
N-Hydroxy-N—((R)-2-{N′-[4-(2,3,4-trimethoxy-phenyl)-pyrimidin-2-yl]-hydrazinocarbonyl}-heptyl)-formamide.
N-{(R)-4,4-Dimethyl-2-[N′-(4-trifluoromethyl-pyrimidin-2-yl)-hydrazinocarbonyl]-heptyl}-N-hydroxy-formamide.
N-{(R)-4,4-Dimethyl-2-[N′-(7-methyl-benzo[1,2,4]triazin-3-yl)-hydrazinocarbonyl]-heptyl}-N-hydroxy-formamide.
N-{(R)-4,4-Dimethyl-2-[N′-(5-methyl-benzo[1,2,4]triazin-3-yl)-hydrazinocarbonyl]-heptyl}-N-hydroxy-formamide.
N-{(R)-4,4-Dimethyl-2-[N′-(4-methyl-pyridin-2-yl)-hydrazinocarbonyl]-heptyl}-N-hydroxy-formamide.
N-Hydroxy-N-{(R)-2-[N′-(6-morpholin-4-yl-pyrimidin-4-yl)-hydrazinocarbonyl]-heptyl}-formamide.
N-Hydroxy-N—[(R)-2-(N′-{5-[4-(2-hydroxy-ethoxy)-phenyl]-[1,2,4]triazin-3-yl}-hydrazinocarbonyl)-heptyl]-formamide.
N-{(R)-2-[N′-(4-Furan-2-yl-pyrimidin-2-yl)-hydrazinocarbonyl]-heptyl}-N-hydroxy-formamide.
N—((R)-2-{N′-[4-(3,5-Dimethyl-isoxazol-4-yl)-pyrimidin-2-yl]-hydrazinocarbonyl}-heptyl)-N-hydroxy-formamide.
N-Hydroxy-N-{(R)-2-[N′-(4-methyl-1-oxy-pyridin-2-yl)-hydrazinocarbonyl]-heptyl}-formamide.
2-(N′-{(R)-2-[(Formyl-hydroxy-amino)-methyl]-heptanoyl}-hydrazino)-6-methyl-nicotinic acid.
N-Hydroxy-N-{(R)-2-[N′-(3-methoxy-pyridin-2-yl)-hydrazinocarbonyl]-heptyl}-formamide.
N-Hydroxy-N-{(2R)-2-[(N′-{4-[4-(methylsulfonyl)phenyl]-pyrimidin-2-yl}-hydrazino)-carbonyl]-heptyl}-formamide.
N-Hydroxy-N-[(2R)-2-({N′-[4-(furan-3-yl)-pyrimidin-2-yl]-hydrazino}-carbonyl)-heptyl]-formamide.
N-[(2R)-2-({N′-[4-(2-aminophenyl)-pyrimidin-2-yl]-hydrazino}-carbonyl)-heptyl]-N-hydroxy-formamide.
N-Hydroxy-N-[(2R)-2-({N′-[5-(5-methyl-1,3,4-oxadiazol-2-yl)-4-(trifluoromethyl)-pyrimidin-2-yl]-hydrazino}-carbonyl)-heptyl]-formamide.
N-Hydroxy-N-((2R)-2-(cyclopentylmethyl)-3-{N′-[5-(5-methyl-1,3,4-oxadiazol-2-yl)-4-(trifluoromethyl)-pyrimidin-2-yl]-hydrazino}-3-oxopropyl)-formamide.
N-[(2R)-2-({N′-[6-(dimethylamino)-2-methyl-pyrimidin-4-yl]-hydrazino}-carbonyl)-heptyl]-N-hydroxy-formamide.
N-[(2R)-2-({N′-[2-Cyclopropyl-6-(dimethylamino)-pyrimidin-4-yl]-hydrazino}-carbonyl)-heptyl]-N-hydroxy-formamide.
N-Hydroxy-N-[(2R)-4-(2-thienyl)-2-({N′-[4-(trifluoromethyl)-pyrimidin-2-yl]-hydrazino}-carbonyl)-butyl]-formamide.
N-Hydroxy-N-[(2R)-2-{[N′-(4-methyl-pyrimidin-2-yl)hydrazino]carbonyl}-4-(2-thienyl)-butyl]-formamide.
N-[(2R)-2-[(N′-{4-Ethyl-6-[ethyl(methyl)amino]-1,3,5-triazin-2-yl}-hydrazino)-carbonyl]-4-(2-thienyl)butyl]-N-hydroxy-formamide.
N-Hydroxy-N-((2R)-3-oxo-2-(2-thienylmethyl)-3-{N′-[4-(trifluoromethyl)-pyrimidin-2-yl]-hydrazino}-propyl)-formamide.
N-Hydroxy-N-[(2R)-3-[N′-(4-methyl-pyrimidin-2-yl)hydrazino]-3-oxo-2-(2-thienylmethyl)-propyl]-formamide.
N-[(2R)-3-(N′-{4-Ethyl-6-[ethyl(methyl)amino]-1,3,5-triazin-2-yl}hydrazino)-3-oxo-2-(2-thienylmethyl)-propyl]-N-hydroxy-formamide.
N-Hydroxy-N-[(2R)-2-({N′-[2-methyl-6-(pyridin-2-yl)-pyrimidin-4-yl]-hydrazino}-carbonyl)-heptyl]-formamide.
N-Hydroxy-N-[(2R)-2-({N′-[6-(pyridin-2-yl-methyl)-pyridazin-3-yl]-hydrazino}-carbonyl)-heptyl]-formamide.
N-Hydroxy-N-[(2R)-2-({N′-[2-methyl-6-(morpholin-4-yl)-pyrimidin-4-yl]-hydrazino}-carbonyl)-heptyl]-formamide.
N-Hydroxy-N-[(2R)-2-({N′-[6-(morpholin-4-yl)-2-(trifluoromethyl)-pyrimidin-4-yl]-hydrazino}-carbonyl)-heptyl]-formamide.
N-Hydroxy-N-{(2R)-2-[(N′-{4-[methyl-(pyridin-2-yl)-amino]-pyrimidin-2-yl}-hydrazino)-carbonyl]-heptyl}-formamide.
N-Hydroxy-N-((2R)-2-(cyclopentylmethyl)-3-{N′-[4-cyclopropyl-6-(dimethylamino)-1,3,5-triazin-2-yl]-hydrazino}-3-oxopropyl)-formamide.
N-Benzo[1,3]dioxol-5-yl-methyl-hydrazinecarboxylic acid tert-butyl ester.
N—[(R)-2-(N′-Benzo[1,3]dioxol-5-yl-methyl-hydrazinocarbonyl)-heptyl]-N-hydroxy-formamide.
N-{(R)-2-[N′-(2,3-Dihydro-[1,4]dioxino[2,3-b]pyridin-7-yl-methyl)-hydrazinocarbonyl]-heptyl}-N-hydroxy-formamide.
N-{(R)-2-[N′-(4-Dimethylamino-benzyl)-hydrazinocarbonyl]-heptyl}-N-hydroxy-formamide.
N-Hydroxy-N—((R)-2-{N′-[2-(5,6,7,8-tetrahydro-[1,8]naphthyridin-2-yl)-ethyl]-hydrazinocarbonyl]-heptyl}-N-hydroxy-formamide.
N-Hydroxy-N—[(R)-2-(N′-quinolin-2-yl-methyl-hydrazinocarbonyl)-heptyl]-formamide.
N-Hydroxy-N-{(R)-2-[N′-(1,2,3,4-tetrahydro-quinolin-2-yl-methyl)-hydrazinocarbonyl]-heptyl}-formamide.
N-Hydroxy-N—[(R)-2-(N′-quinolin-6-yl-methyl-hydrazinocarbonyl)-heptyl]-formamide.
N—[(R)-2-(N′-Benzofuran-2-yl-methyl-hydrazinocarbonyl)-heptyl]-N-hydroxy-formamide.
N—[(R)-2-(N′-Cyclopropylmethyl-hydrazinocarbonyl)-heptyl]-N-hydroxy-formamide.
N-{(R)-2-[N′-(6-Fluoro-4H-benzo[1,3]dioxin-8-yl-methyl)-hydrazinocarbonyl]-heptyl}-N-hydroxy-formamide.
N-Hydroxy-N-{(R)-2-[N′-(4-methoxy-benzyl)-hydrazinocarbonyl]-heptyl}-formamide.
N-Hydroxy-N-{(R)-2-[N′-(2-methoxy-benzyl)-hydrazinocarbonyl]-heptyl}-formamide.
N-Hydroxy-N-{(R)-2-[N′-(tetrahydro-furan-3-yl-methyl)-hydrazinocarbonyl]-heptyl}-formamide.
N—[(R)-2-(N′-Furan-3-yl-methyl-hydrazinocarbonyl)-heptyl]-N-hydroxy-formamide.
N-{(R)-2-[N′-(2,3-Dihydro-benzo[1,4]dioxin-6-yl-methyl)-hydrazinocarbonyl]-heptyl}-N-hydroxy-formamide.
N-{(R)-2-[N′-(2,3-Dihydro-benzo[1,4]dioxin-2-yl-methyl)-hydrazinocarbonyl]-heptyl}-N-hydroxy-formamide.
N-Hydroxy-N-{(R)-2-[N′-(2-phenoxy-ethyl)-hydrazinocarbonyl]-heptyl}-formamide.
N-{(R)-2-[N′-((S)-2,3-Dihydroxy-propyl)-hydrazinocarbonyl]-heptyl}-N-hydroxy-formamide.
N-Hydroxy-N-{(R)-2-[N′-(5-methyl-isoxazol-3-yl-methyl)-hydrazinocarbonyl]-heptyl}-formamide.
N—((R)-2-{N′-[1-(1-Benzo[1,3]dioxol-5-yl-methanoyl)-piperidin-4-yl]-hydrazinocarbonyl}-heptyl)-N-hydroxy-formamide.
N—((R)-2-{N′-[1-(1-Benzofuran-2-yl-methanoyl)-piperidin-4-yl]-hydrazinocarbonyl}-heptyl)-N-hydroxy-formamide.
N-Hydroxy-N—[(R)-2-(N′-{1-[1-(7-methoxy-benzofuran-2-yl)-methanoyl]-piperidin-4-yl}-hydrazinocarbonyl)-heptyl]-formamide.
N-{(R)-2-[N′-(1-Benzyl-piperidin-4-yl)-hydrazinocarbonyl]-heptyl}-N-hydroxy-formamide.
N—[(R)-2-(N′-{1-[1-(3,4-Dichloro-phenyl)-methanoyl]-piperidin-4-yl}-hydrazinocarbonyl)-heptyl]-N-hydroxy-formamide.
N—[(R)-2-(N′-{1-[1-(2,3-Dichloro-phenyl)-methanoyl]-piperidin-4-yl}-hydrazinocarbonyl)-heptyl]-N-hydroxy-formamide.
N-Hydroxy-N—[(R)-2-(N′-{1-[1-(4-methyl-piperazin-1-yl)-methanoyl]-pentyl}-hydrazinocarbonyl)-heptyl]-formamide.
N—[(R)-2-(N′-Benzyl-hydrazinocarbonyl)-heptyl]-N-hydroxy-formamide.

Yet, even more preferred PDF inhibitors within the definition of formula (1) are N-hydroxy-N-{(R)-2-[N′-(4-trifluoromethyl-pyrimidin-2-yl)-hydrazinocarbonyl]-heptyl}-formamide (Compound (1a)), N-hydroxy-N-((2R)-2-(cyclopentylmethyl)-3-{N′-[4-(morpholin-4-yl)-6-(trifluoromethyl)-pyrimidin-2-yl]-hydrazino}-3-oxopropyl)-formamide (Compound (1b)), and N-hydroxy((2R)-2-{[2-(5-methyl-1,2,4-benzotriazin-3-yl)hydrazino]carbonyl}heptyl)formamide (Compound (1c)) as represented by formulas (1a), (1b), and (1c), which are disclosed in WO 2003101442 as Examples 3, 79, and 83, respectively.

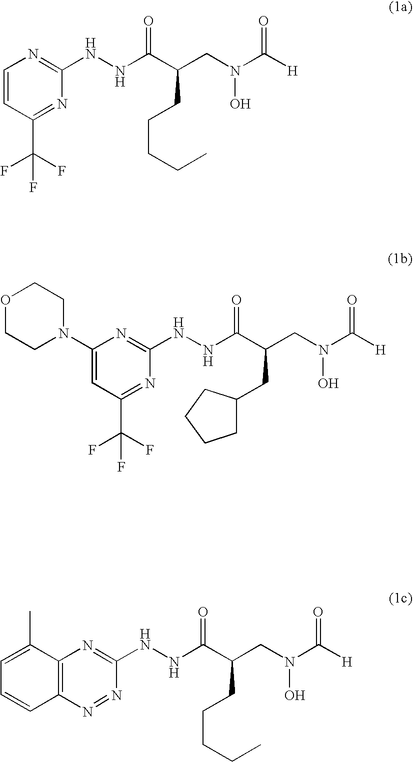

In an additional aspect, another class of preferred PDF inhibitors useful in the present invention are compounds of formula (1) disclosed in WO2002070541, published Sep. 12, 2002, herein renumbered as a compound of formula (2):

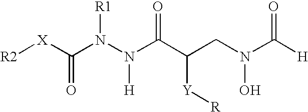

(2) X═O, NR₃ or a bond;
Y═O, CH₂ or a bond
   wherein R, R1, R2, R3, X, and Y are defined as in WO2002070541, i.e.,
   R represents:
C₂₋₆ alkyl (optionally substituted by alkoxy, halogen, or C₁₋₃ alkylsulfanyl), C₂₋₆ alkenyl (optionally substituted by alkoxy, halogen, or C₁₋₃ alkylsulfanyl), C₂₋₆ alkynyl (optionally substituted by alkoxy, halogen, or C₁₋₃ alkylsulfanyl), (CH₂)ₙ—C₃₋₆ carbocycle (optionally substituted by alkoxy, halogen, or C₁₋₃ alkylsulfanyl), (CH₂)ₙ—R4 {where R4 is phenyl, furan, benzofuran, thiophene, benzothiophene, tetrahydrofuran, tetrahydropyran, dioxane, 1,4-benzodioxane or benzo[1,3]dioxole; R4 is optionally substituted by one or more Cl, Br, I, C₁₋₃ alkyl (optionally substituted by one to three F) or C₁₋₂ alkoxy (optionally substituted by one to three F)};
   R1 represents:
hydrogen, C₁₋₆ alkyl (optionally substituted by hydroxy, halogen, amino, guanidino, phenyl, pyridyl, pyrrolyl, indolyl, imidazolyl, furanyl, benzofuranyl, piperidinyl, morpholinyl, quinolinyl, piperazinyl or dimethylaminophenyl) or (CH₂)ₙ—C₃₋₇ carbocycle;
   R2 represents:
hydrogen (provided that X is not 0), C₁₋₃ substituted alkyl, C₂₋₃ substituted alkenyl, C₂₋₃ substituted alkynyl, (CH₂)ₙ—C₃₋₆ substituted carbocycle, aryl, heteroaryl, heterocyclic, carboxy (provided that X is not NR3 or O) or aminocarbonyl (provided that X is not NR3 or O);
   R3 represents:
hydrogen, C₁₋₃ substituted alkyl, phenyl, or may be taken together with R2 and the nitrogen atom to which they are attached to form an optionally substituted heterocyclic ring which is optionally fused to an aryl, a heteroaryl, or a second heterocyclic ring;
   X represents O, NR3 or a covalent bond;
   Y represents O, CH₂ or a covalent bond;
   n=0-2;
   or a salt, solvate, or physiologically functional derivative thereof.

In another aspect, the preferred compounds of formula (2) are in which R1 group is hydrogen. Furthermore, in this invention the most preferred absolute configuration of compounds of the formula (2) is indicated below:

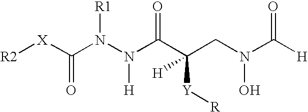

X═O, NR₃ or a bond;
Y═O, CH₂ or a bond

In a further aspect, the preferred compounds of formula (2) are wherein X═O, and R, R1, R2, R3, R4, Y and n are as defined above.

Yet in a further aspect, the preferred compounds of formula (2) are wherein X═NR3, and R, R1, R2, R3, R4, Y and n are as defined above.

There is also provided as another preferred embodiment the compounds of formula (2) wherein X is a covalent bond, and R, R1, R2, R3, R4, Y and n are as defined above.

Even more preferred compounds of formula (2) are selected from the group consisting of:
N-Butyl-N-(t-butoxycarbonyl)-N′-{(2R)-[(formylhydroxyamino)methyl]-heptanoyl}-hydrazine.
N-Butyl-N-phenoxycarbonyl-N′-{(2R)-[(formylhydroxyamino)methyl]-heptanoyl}-hydrazine.
N-Isobutyl-N-(t-butoxycarbonyl)-N′-{(2R)-[(formylhydroxyamino)methyl]-heptanoyl}-hydrazine.
N-Isobutyl-N-phenoxycarbonyl-N′-{(2R)-[(formylhydroxyamino)methyl]-heptanoyl}-hydrazine.
N-Phenethyl-N-(t-butoxycarbonyl)-N′-{2-[(formylhydroxyamino)methyl]-heptanoyl}-hydrazine.
N-Cyclohexylmethyl-N-(t-butoxycarbonyl)-N′-{2-[(formylhydroxyamino)methyl]-heptanoyl}-hydrazine.
N-Benzyl-N-(t-butoxycarbonyl)-N′-{2-[(formylhydroxyamino)methyl]-heptanoyl}-hydrazine.
N-(3-pyridin-3-yl-propyl)-N-(t-butoxycarbonyl)-N′-{2-[(formylhydroxyamino)methyl]-heptanoyl}-hydrazine.
N-(2-Morpholin-4-yl-ethyl)-N-(t-butoxycarbonyl)-N′-{2-[(formylhydroxyamino)methyl]-heptanoyl}-hydrazine.
N-(4-Hydroxy-butyl)-N-(t-butoxycarbonyl)-N′-{2-[(formylhydroxyamino)methyl]-heptanoyl}-hydrazine.
N-(4-Amino-butyl)-N-(t-butoxycarbonyl)-N′-{2-[(formylhydroxyamino)methyl]-heptanoyl}-hydrazine.
N-(Tetrahydro-pyran-4-yl)-N-(t-butoxycarbonyl)-N′-{2-[(formylhydroxyamino)methyl]-heptanoyl}-hydrazine.
N-Methyl-N-(t-butoxycarbonyl)-N′-{2-[(formylhydroxyamino)methyl]-heptanoyl}-hydrazine.
N-(3-Aminopropyl)-N-(t-butoxycarbonyl)-N′-{2-[(formylhydroxyamino)methyl]-heptanoyl}-hydrazine.
N-(t-Butoxycarbonyl)-N′-{(2R)-[(formylhydroxyamino)methyl]-heptanoyl}-hydrazine. N-(3-Hydroxypropyl)-N-(t-butoxycarbonyl)-N′-{2-[(formylhydroxyamino)methyl]-heptanoyl}-hydrazine.
N-Butyl-N-(t-butoxycarbonyl)-N′-{(2S)-[(formylhydroxyamino)methyl]-heptanoyl}-hydrazine.
N-Butyl-N-(phenoxycarbonyl)-N′-{(2S)-[(formylhydroxyamino)methyl]-heptanoyl}-hydrazine.
N-[2-(4-Dimethylaminophenyl)ethyl]-N-(t-butoxycarbonyl)-N′-{2-[(formylhydroxyamino)methyl]-heptanoyl}-hydrazine.
N-(t-Butoxycarbonyl)-N′-{2-[(formylhydroxyamino)methyl]-heptanoyl}-hydrazine.
N-Pentyl-N-(t-butoxycarbonyl)-N′-{2-[(formylhydroxyamino)methyl]-heptanoyl}-hydrazine.
N-[2-(1H-Indol-3-yl)-ethyl]-N-(t-butoxycarbonyl)-N′-{2-[(formylhydroxyamino)methyl]-heptanoyl}-hydrazine.
N-Isopentyl-N-(t-butoxycarbonyl)-N′-{2-[(formylhydroxyamino)methyl]-heptanoyl}-hydrazine.
N-Cyclohexyl-N-(t-butoxycarbonyl)-N′-{2-[(formylhydroxyamino)methyl]-heptanoyl}-hydrazine.
N-(1-Ethyl-propyl)-N-(t-butoxycarbonyl)-N′-{2-[(formylhydroxyamino)methyl]-heptanoyl}-hydrazine.
N-Isopropyl-N-(t-butoxycarbonyl)-N′-{2-[(formylhydroxyamino)methyl]-heptanoyl}-hydrazine.
N-Propyl-N-(t-butoxycarbonyl)-N′-{2-[(formylhydroxyamino)methyl]-heptanoyl}-hydrazine.
N-Ethyl-N-(t-butoxycarbonyl)-N′-{2-[(formylhydroxyamino)methyl]-heptanoyl}-hydrazine.
N-Methoxycarbonyl-N′-{2-[(formylhydroxyamino)methyl]-heptanoyl}-hydrazine.
N-{[1-(3,5-Dimethoxyphenyl)-1-methyl-ethoxy]carbonyl}-N′-{2-[(formylhydroxyamino)methyl]-heptanoyl}-hydrazine.

Many PDF inhibitors are already known. As stated earlier, any PDF inhibitor can be used for the present invention. Other exemplary PDF inhibitors are described in, for example:
WO 2004052919 A2 published Jun. 24, 2004
WO 2003104209 A1 published Dec. 18, 2003
WO 2003077913 A1 published Sep. 25, 2003
WO 2003002522 A1 published Jan. 9, 2003
WO 2002098901 A2 published Dec. 12, 2002
WO 2002081426 A1 published Oct. 17, 2002
WO 2002070654 A2 published Sep. 12, 2002
WO 2002070653 A2 published Sep. 12, 2002
WO 2002070540 A2 published Sep. 12, 2002
WO 2001085170 A1 published Nov. 15, 2001
WO 2001085160 A1 published Nov. 15, 2001
Waller, Andrew S.; Clements, John M. Novel approaches to antimicrobial therapy: peptide deformylase. Current Opinion in Drug Discovery & Development (2002), 5(5), 785-792.
Clements, John M.; Ayscough, Andrew P.; Keavey, Kenneth; East, Stephen P. Peptide deformylase inhibitors, potential for a new class of broad spectrum antibacterials. Current Medicinal Chemistry: Anti-Infective Agents (2002), 1(3), 239-249.
Yuan, Z.; Trias, J.; White, R. J. Deformylase as a novel antibacterial target. Drug Discovery Today (2001), 6(18), 954-961.
Pei, Dehua. Peptide deformylase: A target for novel antibiotics? Emerging Therapeutic Targets (2001), 5(1), 23-40.
Giglione, Carmela; Pierre, Michele; Meinnel, Thierry. Peptide deformylase as a target for new generation, broad spectrum antimicrobial agents. Molecular Microbiology (2000), 36(6), 1197-1205.
Jones, Ronald N.; Fritsche, Thomas R.; Sadera, Helio S. Antimicrobial spectrum and activity of NVP PDF-713, a novel peptide deformylase inhibitor, tested against 1,837 recent gram-positive clinical isolates. Diagnostic Microbiology and Infectious Disease (2004), 49(1), 63-65.
WO 2004087643 A1 published Oct. 14, 2004
WO 2004087133 A1 published Oct. 14, 2004
WO 2004076053 A2 published Sep. 10, 2004
WO 2004026824 A1 published Apr. 1, 2004
WO 2004018453 A1 published Apr. 3, 2004
WO 2002102790 A1 published Dec. 27, 2002
WO 2002102791 A1 published Dec. 27, 2002
Nguyen, Kiet T.; Hu, Xubo; Pei, Dehua. Slow-binding inhibition of peptide deformylase by cyclic peptidomimetics as revealed by a new spectrophotometric assay. Bioorganic Chemistry (2004), 32(3), 178-191.
Lofland, Denene; Difuntorum, Stacey; Waller, Andrew; Clements, John M.; Weaver, Mellany K.; Karlowsky, James A.; Johnson, Kirk. In vitro antibacterial activity of the peptide deformylase inhibitor BB-83698. Journal of Antimicrobial Chemotherapy (2004), 53(4), 664-668.
Gross, M.; Clements, J.; Beckett, R. P.; Thomas, W.; Taylor, S.; Lofland, D.; Ramanathan-Girish, S.; Garcia, M.; Difuntorum, S.; Hoch, U.; Chen, H.; Johnson, K. W. Oral anti-pneumococcal activity and pharmacokinetic profiling of a novel peptide deformylase inhibitor. Journal of Antimicrobial Chemotherapy (2004), 53(3), 487-493.
Molteni, Valentina; He, Xiaohui; Nabakka, Juliet; Yang, Kunyong; Kreusch, Andreas; Gordon, Perry; Bursulaya, Badry; Warner, Ian; Shin, Tanya; Biorac, Tanya; Ryder, Neil S.; Goldberg, Ron; Doughty, John; He, Yun. Identification of novel potent bicyclic peptide deformylase inhibitors. Bioorganic & Medicinal Chemistry Letters (2004), 14(6), 1477-1481.
WO 2004050638 A1 published Jun. 17, 2004
Coats, Reed A.; Lee, Sheng-Lian; Davis, Kari A.; Patel, Kanu M.; Rhoads, Elaine K.; Howard, Michael H. Stereochemical Definition and Chirospecific Synthesis of the Peptide Deformylase Inhibitor Sch 382583. Journal of Organic Chemistry (2004), 69(5), 1734-1737.
Chen, D.; Hackbarth, C.; Ni, Z. J.; Wu, C.; Wang, W.; Jain, R.; He, Y.; Bracken, K.; Weidmann, B.; Patel, D. V.; Trias, J.; White, R. J.; Yuan, Z. Peptide deformylase inhibitors as antibacterial agents: Identification of VRC3375, a proline-3-alkylsuccinyl hydroxamate derivative, by using an integrated combinatorial and medicinal chemistry approach. Antimicrobial Agents and Chemotherapy (2004), 48(1), 250-261.
East, Stephen P.; Beckett, R. Paul; Brookings, Daniel C.; Clements, John M.; Doel, Sheila; Keavey, Kenneth; Pain, Gilles; Smith, Helen K.; Thomas, Wayne; Thompson, Alison J.; Todd, Richard S.; Whittaker, Mark. Peptide deformylase inhibitors with activity against respiratory tract pathogens. Bioorganic & Medicinal Chemistry Letters (2004), 14(1), 59-62.
Smith, Helen K.; Beckett, R. Paul; Clements, John M.; Doel, Sheila; East, Stephen P.; Launchbury, Steven B.; Pratt, Lisa M.; Spavold, Zoe M.; Thomas, Wayne; Todd, Richard S.; Whittaker, Mark. Structure-activity relationships of the peptide deformylase inhibitor BB-3497: modification of the metal binding group. Bioorganic & Medicinal Chemistry Letters (2002), 12(24), 3595-3599.
Hackbarth, Corinne J.; Chen, Dawn Z.; Lewis, Jason G.; Clark, Kirk; Mangold, James B.; Cramer, Jeffrey A.; Margolis, Peter S.; Wang, Wen; Koehn, Jim; Wu, Charlotte; Lopez, S.; Withers, George, III; Gu, Helen; Dunn, Elina; Kulathila, R.; Pan, Shi-Hao; Porter, Wilma L.; Jacobs, Jeff; Trias, Joaquim; Patel, Dinesh V.; Weidmann, Beat; White, Richard J.; Yuan, Zhengyu. N-alkyl urea hydroxamic acids as a new class of peptide deformylase inhibitors with antibacterial activity.
Antimicrobial Agents and Chemotherapy (2002), 46(9), 2752-2764.
WO 2001044179 A1 published Jun. 21, 2001
WO 2001044178 A1 published Jun. 21, 2001
Thorarensen, A.; Douglas, M. R., Jr.; Rohrer, D. C.; Vosters, A. F.; Yem, A. W.; Marshall, V. D.; Lynn, J. C.; Bohanon, M. J.; Tomich, P. K.; Zurenko, G. E.; Sweeney, M. T.; Jensen, R. M.; Nielsen, J. W.; Seest, E. P.; Dolak, L. A. Identification of novel potent hydroxamic acid inhibitors of peptidyl deformylase and the importance of the hydroxamic acid functionality on inhibition. Bioorganic & Medicinal Chemistry Letters (2001), 11(11), 1355-1358.
Clements, John M.; Beckett, R. Paul; Brown, Anthony; Catlin, Graham; Lobell, Mario; Palan, Shilpa; Thomas, Wayne; Whittaker, Mark; Wood, Stephen; Salama, Sameeh; Baker, Patrick J.; Rodgers, H. Fiona; Barynin, Vladimir; Rice, David W.; Hunter, Michael G. Antibiotic activity and characterization of BB-3497, a novel peptide deformylase inhibitor. Antimicrobial Agents and Chemotherapy (2001), 45(2), 563-570.
Apfel, Christian; Banner, David W.; Bur, Daniel; Dietz, Michel; Hirata, Takahiro; Hubschwerlen, Christian; Locher, Hans; Page, Malcolm G. P.; Pirson, Wolfgang; Rosse, Gerard; Specklin, Jean-Luc. Hydroxamic acid derivatives as potent peptide deformylase inhibitors and antibacterial agents. Journal of Medicinal Chemistry (2000), 43(12), 2324-2331.
Green, Barbara Gordon; Toney, Jeffrey H.; Kozarich, John W.; Grant, Stephan K. Inhibition of bacterial peptide deformylase by biaryl acid analogs. Archives of Biochemistry and Biophysics (2000), 375(2), 355-358.
Chen, Dawn Z.; Patel, Dinesh V.; Hackbarth, Corinne J.; Wang, Wen; Dreyer, Geoffrey; Young, Dennis C.; Margolis, Peter S.; Wu, Charlotte; Ni, Zi-Jie; Trias, Joaquim; White, Richard J.; Yuan, Zhengyu. Actinonin, a naturally occurring antibacterial agent, is a potent deformylase inhibitor. Biochemistry (2000), 39(6), 1256-1262.

All patents, patent publications, and literature references cited herein are incorporated by reference in their entirety.

Macrolide antibiotics of the present invention are defined as compounds having 14-,15-, or 16-membered lactone rings with one or more deoxy sugars attached. Many such macrolide antibiotics are known. More preferred macrolide antibiotics are described in Retsema, J and Fu, W. (2001) Int. J. Antimicrob. Agents, 18, S3-S10 and Kanfer, I., Skinner, M. F. and Walker, R. B. (1998) J. Chromatogr., 812, 255-286. Even more preferred macrolide antibiotics for the purpose of the present invention are selected from the group consisting of erythromycin, azithromycin, tylosin, oleandomycin, roxithromycin, dirithromycin, clarithromycin, flurithromycin, josamycin, rosaramicin, rokitamycin, kitasamycin, mirosamycin, spiramycin, and carbomycin.

### COMPOSITIONS, ADMINISTRATION AND BIOLOGICAL ASSAYS

The present invention contemplates co-administration of a PDF inhibitor (or a pharmaceutically acceptable salt, solvate, or physiologically functional derivative thereof) and a macrolide antibiotic (or a pharmaceutically acceptable salt, solvate, or physiologically functional derivative thereof) in separate formulations or in a single composition containing both a PDF inhibitor (or a pharmaceutically acceptable salt, solvate, or physiologically functional derivative thereof) and a macrolide antibiotic (or a pharmaceutically acceptable salt, solvate, or physiologically functional derivative thereof).

A formulation (composition) containing a PDF inhibitor (or a pharmaceutically acceptable salt, solvate, or physiologically functional derivative thereof) and/or a macrolide antibiotic (or a pharmaceutically acceptable salt, solvate, or physiologically functional derivative thereof) may be administered in a standard manner for antibiotics, for example orally, parenterally, sub-lingually, dermally, transdermally, rectally, via inhalation or via buccal administration.

A composition containing a PDF inhibitor (or a pharmaceutically acceptable salt, solvate, or physiologically functional derivative thereof) and/or a macrolide antibiotic (or a pharmaceutically acceptable salt, solvate, or physiologically functional derivative thereof) when given orally can be formulated as syrups, tablets, capsules, creams and lozenges. A syrup formulation will generally consist of a suspension or solution of the compound(s) or salt(s) in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example, using the aforementioned carriers in a hard gelatin capsule shell. Where the composition is in the form of a soft gelatin shell capsule, any pharmaceutical carrier routinely used for preparing dispersions or suspensions may be considered, for example, aqueous gums, celluloses, silicates or oils, and incorporated in a soft gelatin capsule shell.

Typical parenteral compositions consist of a solution or suspension of a PDF inhibitor (or a pharmaceutically acceptable salt, solvate, or physiologically functional derivative thereof) and/or a macrolide antibiotic (or a pharmaceutically acceptable salt, solvate, or physiologically functional derivative thereof) in a sterile aqueous or non-aqueous carrier optionally containing a parenterally acceptable oil, for example, polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a PDF inhibitor (or a pharmaceutically acceptable salt, solvate, or physiologically functional derivative thereof) and/or a macrolide antibiotic (or a pharmaceutically acceptable salt, solvate, or physiologically functional derivative thereof) with a binding and/or lubricating agent, for example, polymeric glycols, gelatins, cocoa-butter or other low melting vegetable waxes or fats or their synthetic analogs.

Typical dermal and transdermal formulations comprise a conventional aqueous or non-aqueous vehicle, for example, a cream, ointment, lotion or paste or are in the form of a medicated plaster, patch or membrane.

Preferably the composition is in unit dosage form, for example a tablet, capsule or metered aerosol dose, so that the patient may administer a single dose.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5th Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of formula (1) or (2) or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in the compound of formula (1) or (2). Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Each dosage unit for oral administration contains suitable amounts, from 0.1 mg to 500 mg/Kg, and preferably from 1 mg to 100 mg/Kg, of a PDF inhibitor and a macrolide antibiotic, independently, or pharmaceutically acceptable salts, solvates or physiologically functional derivatives thereof. Each dosage unit for parenteral administration contains suitable amounts, from 0.1 mg to 100 mg/Kg, of a PDF inhibitor and a macrolide antibiotic, independently, or pharmaceutically acceptable salts, solvates or physiologically functional derivatives thereof. Each dosage unit for intranasal administration contains suitable amounts, 1-400 mg and preferably 10 to 200 mg per person, of a PDF inhibitor and a macrolide antibiotic, independently, or pharmaceutically acceptable salts, solvates or physiologically functional derivatives thereof. A topical formulation contains suitably 0.01 to 5.0% of a PDF inhibitor and a macrolide antibiotic, independently, or pharmaceutically acceptable salts, solvates or physiologically functional derivatives thereof.

The pharmaceutical formulation and co-administration (of a PDF inhibitor and a macrolide antibiotic) as discussed above relate to the treatment of all bacterial infections, including, but not limited to, the genera of Streptococcus, Staphylococcus, Mycoplasma, Mycobacterium, Haemophilus, Moraxella, Escherichia, Salmonella, Klebsiella, Legionella, Chliamydia, Pseudomonas, Helicobacter, Neisseria, Proteus, Yersinia, Brucella, Borrelia, Treponema, Enterobacter, and Bordetella, and in particular, Streptococcus pneumoniae, Staphylococcus aureus and Haemophilus influenzae.

A dosage unit may be administered from 1 to 6 times a day, sufficient to exhibit the desired activity.

No unacceptable toxicological effects are expected when compounds of the present invention are administered in accordance with the present invention.

The biological activity of the compounds within the scope of the invention are demonstrated by the following examples:

### EXAMPLE 1

### Subinhibitory Concentrations of PDF Inhibitors Decrease the MICs of Macrolides against S. pneumoniae 1629

| | MIC (ug/ml) [fold decrease MIC] | | | | |
|---|---|---|---|---|---|
| | | Antibiotic with | | Antibiotic with | |
| | | 0.06 ug/mL | | 0.125 ug/mL | |
| | Antibiotic | (¼ MIC) | | (½ MIC) | |
| Antibiotic | alone | of Compound (1b)* | | of Compound (1b)* | |
| Erythromycin | 0.03 | 0.008 | [4] | 0.002 | [16] |
| Azithromycin | 0.03 | 0.016 | [2] | 0.004 | [8] |
| Ciprofloxacin | 0.5 | 0.5 | [0] | 0.5 | [0] |
| Mupirocin | 0.5 | 0.5 | [0] | 0.5 | [0] |

*MIC of compound (1b) = 0.25 ug/ml

The experiment showed that
1. Subinhibitory concentrations of Compound (1b) decreased the MIC of erythromycin against S. pneumoniae by 4-16 fold.
2. Subinhibitory concentrations of Compound (1b) decreased the MIC of azithromycin against S. pneumoniae by 2-8 fold.
3. Subinhibitory concentrations of Compound (1b) had no effect on the antibacterial potency of other antibiotics, like ciprofloxacin or mupirocin.

### EXAMPLE 2

### Subinhibitory Concentrations of Macrolides Decrease the MICs of PDF Inhibitors Against S. pneumoniae 1629

| | | | Compound (1b) MIC | |
|---|---|---|---|---|
| | | | (μg/ml) | |
| Antibiotic concentration (μg/ml) | | | [fold decrease MIC] | |
| None | | | 0.25 | |
| Erythromycin | 0.008 | {¼ × MIC} | 0.03 | [8] |
| (MIC, 0.03 μg/ml) | 0.016 | {½ × MIC} | 0.008 | [32] |
| Azithromycin | 0.008 | {¼ × MIC} | 0.06 | [4] |
| (MIC, 0.03 μg/ml) | 0.016 | {½ × MIC} | 0.016 | [16] |
| Ciprofloxacin | 0.125 | {¼ × MIC} | 0.25 | [0] |
| (MIC, 0.5 μg/ml) | 0.25 | {½ × MIC} | 0.25 | [0] |
| Mupirocin | 0.125 | {¼ × MIC} | 0.25 | [0] |
| (MIC, 0.5 μg/ml) | 0.25 | {½ × MIC} | 0.25 | [0] |

The experiment showed that
1. Subinhibitory concentrations of erythromycin decrease the MIC of Compound (1 b) against S. pneumoniae by 8-32 fold.
2. Subinhibitory concentrations of azithromycin decrease the MIC of Compound (1b) against S. pneumoniae by 4-16 fold
3. Subinhibitory concentrations of other antibiotics, like ciprofloxacin or mupirocin, had no effect on the antibacterial potency of the PDF inhibitor.

### EXAMPLE 3

### Subinhibitory Concentrations of PDF Inhibitors Decrease the MICs of Macrolides against S. aureus WCUH29

| | MIC (ug/ml) [fold decrease MIC] | | | | |
|---|---|---|---|---|---|
| | | Antibiotic with | | Antibiotic with | |
| | Antibiotic | 0.5 ug/mL (¼ MIC) | | 1 ug/mL (½ MIC) | |
| Antibiotic | alone | of Compound (1a)* | | of Compound (1a)* | |
| Erythromycin | 0.25 | 0.125 | [2] | 0.016 | [16] |
| Azithromycin | 1 | 0.125 | [8] | 0.03 | [32] |
| Tylosin | 1 | 0.25 | [4] | 0.125 | [8] |
| Ciprofloxacin | 0.25 | 0.25 | [0] | 0.25 | [0] |
| Mupirocin | 0.125 | 0.125 | [0] | 0.125 | [0] |

*MIC of Compound (1a) = 2 ug/mL

The experiment showed that
1. Subinhibitory concentrations of Compound (1a) decreased the MIC of erythromycin against S. aureus by 2-16 fold.
2. Subinhibitory concentrations of Compound (1a) decreased the MIC of azithromycin against S. aureus by 8-32 fold
3. Subinhibitory concentrations of Compound (1a) decreased the MIC of tylosin against S. aureus by 4-8 fold.
4. Subinhibitory concentrations of Compound (1a) had no effect on the antibacterial potency of other antibiotics, like ciprofloxacin or mupirocin.

### EXAMPLE 4

### Subinhibitory Concentrations of Macrolides Decrease the MICs of PDF Inhibitors against S. aureus WCUH29

| | | | Compound (1a) MIC | |
|---|---|---|---|---|
| | | | (μg/ml) | |
| Antibiotic concentration (μg/ml) | | | [fold decrease MIC] | |
| None | | | 2 | |
| Erythromycin | 0.06 | {¼ × MIC} | 0.5 | [4] |
| (MIC, 0.25 μg/ml) | 0.125 | {½ × MIC} | 0.25 | [8] |
| Azithromycin | 0.25 | {¼ × MIC} | 0.5 | [4] |
| (MIC, 1 μg/ml) | 0.5 | {½ × MIC} | 0.25 | [8] |
| Tylosin | 0.25 | {¼ × MIC} | 0.5 | [4] |
| (MIC, 1 μg/ml) | 0.5 | {½ × MIC} | 0.06 | [32] |
| Ciprofloxacin | 0.06 | {¼ × MIC} | 2 | [0] |
| (MIC, 0.25 μg/ml) | 0.125 | {½ × MIC} | 2 | [0] |

The experiment showed that
1. Subinhibitory concentrations of erythromycin and azithromycin decreased the MIC of Compound (1a) against S. aureus by 4-8 fold.
2. Subinhibitory concentrations of tylosin decreased the MIC of Compound (1a) against S. aureus by 4-32 fold.
3. Subinhibitory concentrations of other antibiotics, like ciprofloxacin, had no effect on the antibacterial potency of the PDF inhibitor.

### EXAMPLE 5

### Subinhibitory Concentrations of PDF Inhibitors Decrease the MICs of Macrolides against Representative S. aureus Strains

| | Azithromycin MIC (ug/ml) [fold decrease MIC] | | | | | Tylosin MIC (ug/ml) [fold decrease MIC] | | | | |
|---|---|---|---|---|---|---|---|---|---|---|
| | | +Compound (1a) | | | | | +Compound (1a) | | | |
| S. aureus strain | −Compound (1a) | [¼ × MIC] | | [½ × MIC] | | −Compound (1a) | [¼ × MIC] | | [½ × MIC] | |
| Smith | 1 | 0.25 | [4] | 0.125 | [8] | 1 | 0.25 | [4] | 0.125 | [8] |
| RN4220 | 1 | 0.25 | [4] | 0.125 | [8] | 1 | 0.125 | [8] | 0.06 | [16] |
| Oxford | 0.5 | 0.25 | [2] | 0.06 | [8] | 0.5 | 0.06 | [8] | 0.03 | [16] |
| PK1 | >64 | 32 | [>4] | 16 | [>8] | 2 | 0.25 | [8] | 0.06 | [32] |

The experiment showed that
1. Subinhibitory concentrations of Compound (1a) decreased the MIC of azithromycin against representative S. aureus strains by 2->8 fold.
2. Subinhibitory concentrations of Compound (1a) decreased the MIC of tylosin against representative S. aureus strains by 4-32 fold.

### EXAMPLE 6

### Subinhibitory Concentrations of Macrolides Decrease the MICs of PDF Inhibitors against Representative S. aureus Strains

| | MIC (ug/ml) [fold decrease MIC] | | | | |
|---|---|---|---|---|---|
| | | Compound (1a) | | Compound (1a) | |
| S. aureus | Compound (1a) | with ¼ MIC of | | with ½ MIC of | |
| strain | alone | Tylosin | | Tylosin | |
| Smith | 4 | 0.5 | [8] | 0.125 | [32] |
| RN4220 | 4 | 0.25 | [16] | 0.03 | [128] |
| Oxford | 2 | 1 | [2] | 0.25 | [8] |
| PK1 | 0.5 | 0.06 | [8] | 0.016 | [32] |

The experiment showed that subinhibitory concentrations of tylosin decreased the MIC of Compound (1a) against representative S. aureus strains by 2-128 fold.

### EXAMPLE 7

### Subinhibitory Concentrations of PDF Inhibitors Decrease the MICs of Macrolides against H. influenzae Q1

| | MIC (ug/ml) [fold decrease MIC] | | | | |
|---|---|---|---|---|---|
| | | Antibiotic with | | Antibiotic with | |
| | Antibiotic | 0.5 ug/mL (¼ MIC) | | 1 ug/mL (½ MIC) | |
| Antibiotic | alone | of Compound (1a)* | | of Compound (1a)* | |
| Erythromycin | 8 | 2 | [4] | 1 | [8] |
| Azithromycin | 1 | 0.25 | [4] | 0.125 | [8] |
| Tylosin | 16 | 8 | [2] | 4 | [4] |
| Ciprofloxacin | 0.008 | 0.008 | [0] | 0.008 | [0] |

*MIC of Compound (1a) = 2 ug/mL

The experiment showed that
1. Subinhibitory concentrations of Compound (1a) decreased the MIC of erythromycin and azithromycin against H. influenzae by 4-8 fold.
2. Subinhibitory concentrations of Compound (1a) decreased the MIC of tylosin against H. influenzae by 2-4 fold.
3. Subinhibitory concentrations of Compound (1a) had no effect on the antibacterial potency of other antibiotics, like ciprofloxacin.

### EXAMPLE 8

Subinhibitory Concentrations of Macrolides Decrease the MICs of PDF Inhibitors against H. influenzae Q1

| | MIC (ug/ml) [fold decrease MIC] | | | | |
|---|---|---|---|---|---|
| | | Antibiotic with | | Antibiotic with | |
| | | 0.25 ug/m | | 0.5 ug/mL | |
| | Antibiotic | (¼ MIC) | | (½ MIC) | |
| Antibiotic | alone | of Azithromycin* | | of Azithromycin* | |
| Compound (1a) | 2 | 1 | [2] | 0.5 | [4] |
| Compound (1c) | 1 | 0.5 | [2] | 0.125 | [8] |

*MIC of Azithromycin = 1 ug/ml

The experiment showed that subinhibitory concentrations of azithromycin decreased the MIC of PDF inhibitors against H. influenzae by 2-8 fold.

1. A method of treating bacterial infections in a subject in need thereof comprising administering an effective amount of a PDF inhibitor (or a pharmaceutically acceptable salt, solvate, or physiologically functional derivative thereof) and a macrolide antibiotic (or a pharmaceutically acceptable salt, solvate, or physiologically functional derivative thereof).

2. The method of claim 1 in which the PDF inhibitor is a compound of formula (1): 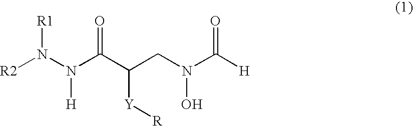
wherein:
R is selected from the group consisting of:
C₂₋₆ alkyl (optionally substituted by alkoxy, halogen, or C₁₋₃ alkylsulfanyl);
C₂₋₆ alkenyl (optionally substituted by alkoxy, halogen, or C₁₋₃ alkylsulfanyl);
C₂₋₆ alkynyl (optionally substituted by alkoxy, halogen, or C₁₋₃ alkylsulfanyl);
(CH₂)ₙ—C₃₋₆ carbocycle (optionally substituted by alkoxy, halogen, or C₁₋₃ alkylsulfanyl); and (CH₂)ₙ—R4, wherein R4 is selected from the group consisting of phenyl, furan, benzofuran, thiophene, benzothiophene, tetrahydrofuran, tetrahydropyran, dioxane, 1,4-benzodioxane or benzo[1,3]dioxole; R4 is optionally substituted by one or more substituent selected from Cl, Br, I, C₁₋₃ alkyl (optionally substituted by one to three F) and
C₁₋₂ alkoxy (optionally substituted by one to three F));
R1 and R2 are independently selected from the group consisting of:
hydrogen, C₁₋₃ substituted alkyl, C₂₋₃ substituted alkenyl, C₂₋₃ substituted alkynyl, (CH₂)ₙ—C₃₋₆ substituted carbocycle, aryl, heteroaryl, and heterocyclic;
Y represents O, CH₂ or a covalent bond; and
n is an integer from 0 to 2.

3. The method of claim 2, wherein R2 of formula (1) represents hydrogen.

4. The method according to claim 3, wherein the compound has the following absolute configuration:

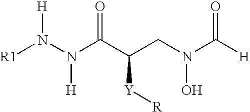

5. The method of claim 4 in which the compound is of the 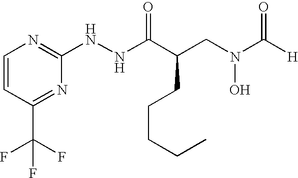
formula

6. The method of claim 4 in which the compound is of the formula

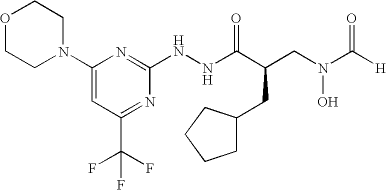

7. The method of claim 4 in which the compound is of the formula

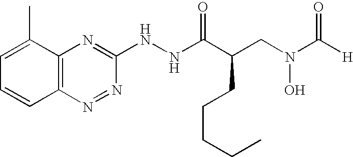

8. The method of claim 1 in which the PDF inhibitor is a compound of formula (2) 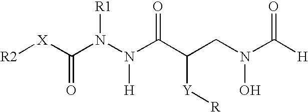
(2) X═O, NR₃ or a bond;
Y═O, CH₂ or a bond
wherein:
R represents:
C₂₋₆ alkyl (optionally substituted by alkoxy, halogen, or C₁₋₃ alkylsulfanyl), C₂₋₆ alkenyl (optionally substituted by alkoxy, halogen, or C₁₋₃ alkylsulfanyl), C₂₋₆ alkynyl (optionally substituted by alkoxy, halogen, or C₁₋₃ alkylsulfanyl), (CH₂)ₙ—C₃₋₆ carbocycle (optionally substituted by alkoxy, halogen, or C₁₋₃ alkylsulfanyl), (CH₂)ₙ—R4 {where R4 is phenyl, furan, benzofuran, thiophene, benzothiophene, tetrahydrofuran, tetrahydropyran, dioxane, 1,4-benzodioxane or benzo[1,3]dioxole; R4 is optionally substituted by one or more Cl, Br, I, C₁₋₃ alkyl (optionally substituted by one to three F) or C₁₋₂ alkoxy (optionally substituted by one to three F)};
R1 represents:
hydrogen, C₁₋₆ alkyl (optionally substituted by hydroxy, halogen, amino, guanidino, phenyl, pyridyl, pyrrolyl, indolyl, imidazolyl, furanyl, benzofuranyl, piperidinyl, morpholinyl, quinolinyl, piperazinyl or dimethylaminophenyl) or (CH₂)ₙ—C₃₋₇ carbocycle;
R2 represents:
hydrogen (provided that X is not 0), C₁₋₃ substituted alkyl, C₂₋₃ substituted alkenyl, C₂₋₃ substituted alkynyl, (CH₂)ₙ—C₃₋₆ substituted carbocycle, aryl, heteroaryl, heterocyclic, carboxy (provided that X is not NR3 or O) or aminocarbonyl (provided that X is not NR3 or O);
R3 represents:
hydrogen, C₁₋₃ substituted alkyl, phenyl, or may be taken together with R2 and the nitrogen atom to which they are attached to form an optionally substituted heterocyclic ring which is optionally fused to an aryl, a heteroaryl, or a second heterocyclic ring;
X represents O, NR3 or a covalent bond;
Y represents O, CH₂ or a covalent bond;
n=0-2.

9. The method of claim 8 in which R1 of the compound of formula (2) is hydrogen.

10. The method of claim 8 in which the compound of formula (2) has the absolute configuration

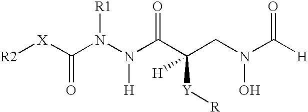

11. The method of claim 1 in which a macrolide antibiotic is selected from the groups consisting of erthromycin, azithromycin tylosin, oleandomycin, roxithromycin, dirithromycin, clarithromycin, flurithromycin, josamycin, rosaramicin, rokitamycin, kitasamycin, mirosamycin, spiramycin and carbomycin.

12. A pharmaceutical composition comprising an effective amount of a PDF inhibitor (or a pharmaceutically acceptable salt, solvate, or physiologically functional derivative thereof) and a macrolide antibiotic (or a pharmaceutically acceptable salt, solvate, or physiologically functional derivative thereof) for treating bacterial infection.

13. The pharmaceutical composition of claim 12 in which the PDF inhibitor is a compound of formula (1): 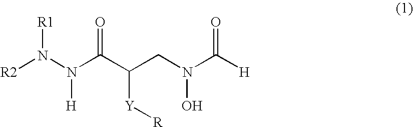
wherein:
R is selected from the group consisting of:
C₂₋₆ alkyl (optionally substituted by alkoxy, halogen, or C₁₋₃ alkylsulfanyl);
C₂₋₆ alkenyl (optionally substituted by alkoxy, halogen, or C₁₋₃ alkylsulfanyl);
C₂₋₆ alkynyl (optionally substituted by alkoxy, halogen, or C₁₋₃ alkylsulfanyl);
(CH₂)ₙ—C₃₋₆ carbocycle (optionally substituted by alkoxy, halogen, or C₁₋₃ alkylsulfanyl); and (CH₂)ₙ—R4, wherein R4 is selected from the group consisting of phenyl, furan, benzofuran, thiophene, benzothiophene, tetrahydrofuran, tetrahydropyran, dioxane, 1,4-benzodioxane or benzo[1,3]dioxole; R4 is optionally substituted by one or more substituent selected from Cl, Br, I, C₁₋₃ alkyl (optionally substituted by one to three F) and C₁₋₂ alkoxy (optionally substituted by one to three F)};
R1 and R2 are independently selected from the group consisting of:
hydrogen, C₁₋₃ substituted alkyl, C₂₋₃ substituted alkenyl, C₂₋₃ substituted alkynyl, (CH₂)ₙ—C₃₋₆ substituted carbocycle, aryl, heteroaryl, and heterocyclic;
Y represents O, CH₂ or a covalent bond; and
n is an integer from 0 to 2.

14. The pharmaceutical composition of claim 13 wherein R2 of formula (1) represents hydrogen.

15. The pharmaceutical composition of claim 13 wherein the compound has the following absolute configuration:

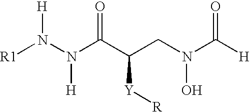

16. The pharmaceutical composition of claim 13 in which the compound is of the formula

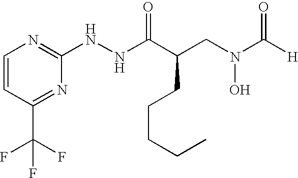

17. The pharmaceutical composition of claim 13 in which the compound is of the formula

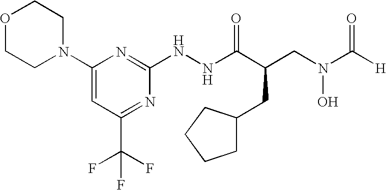

18. The pharmaceutical composition of claim 13 in which the compound is of the formula

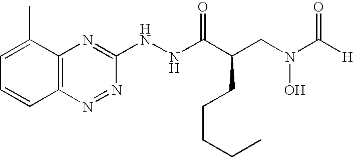

19. The pharmaceutical composition of claim 12 in which the PDF inhibitor is a compound of formula (2) 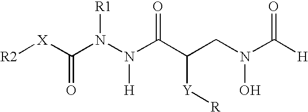
(2) X═O, NR₃ or a bond;
Y═O, CH₂ or a bond
wherein:
R represents:
C₂₋₆ alkyl (optionally substituted by alkoxy, halogen, or C₁₋₃ alkylsulfanyl), C₂₋₆ alkenyl (optionally substituted by alkoxy, halogen, or C₁₋₃ alkylsulfanyl), C₂₋₆ alkynyl (optionally substituted by alkoxy, halogen, or C₁₋₃ alkylsulfanyl), (CH₂)ₙ—C₃₋₆ carbocycle (optionally substituted by alkoxy, halogen, or C₁₋₃ alkylsulfanyl), (CH₂)ₙ—R4 {where R4 is phenyl, furan, benzofuran, thiophene, benzothiophene, tetrahydrofuran, tetrahydropyran, dioxane, 1,4-benzodioxane or benzo[1,3]dioxole; R4 is optionally substituted by one or more Cl, Br, I, C₁₋₃ alkyl (optionally substituted by one to three F) or C₁₋₂ alkoxy (optionally substituted by one to three F)};
R1 represents:
hydrogen, C₁₋₆ alkyl (optionally substituted by hydroxy, halogen, amino, guanidino, phenyl, pyridyl, pyrrolyl, indolyl, imidazolyl, furanyl, benzofuranyl, piperidinyl, morpholinyl, quinolinyl, piperazinyl or dimethylaminophenyl) or (CH₂)ₙ—C₃₋₇ carbocycle;
R2 represents:
hydrogen (provided that X is not 0), C₁₋₃ substituted alkyl, C₂₋₃ substituted alkenyl, C₂₋₃ substituted alkynyl, (CH₂)ₙ—C₃₋₆ substituted carbocycle, aryl, heteroaryl, heterocyclic, carboxy (provided that X is not NR3 or O) or aminocarbonyl (provided that X is not NR3 or O);
R3 represents:
hydrogen, C₁₋₃ substituted alkyl, phenyl, or may be taken together with R2 and the nitrogen atom to which they are attached to form an optionally substituted heterocyclic ring which is optionally fused to an aryl, a heteroaryl, or a second heterocyclic ring;
X represents O, NR3 or a covalent bond;
Y represents O, CH₂ or a covalent bond;
n=0-2.

20. The pharmaceutical composition of claim 19 in which the compound has the absolute configuration:

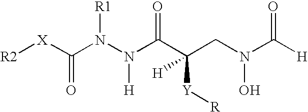

21. The pharmaceutical composition of claim 20 in which R1 is hydrogen.

22. The pharmaceutical composition of claim 12 in which the macrolide antibiotic is selected from the groups consisting of erthromycin, azithromycin, tylosin, oleandomycin, roxithromycin, dirithromycin, clarithromycin, flurithromycin, josamycin, rosaramicin, rokitamycin, kitasamycin, mirosamycin, spiramycin and carbomycin.

23. The method of claim 1 wherein the bacterial infection is caused by any one of the organisms from the genera Streptococcus, Staphylococcus, Mycoplasma, Mycobacterium, Haemophilus, Moraxella, Escherichia, Salmonella, Klebsiella, Legionella, Chiamydia, Pseudomonas, Helicobacter, Neisseria, Proteus, Yersinia, Brucella, Borrelia, Treponema, Enterobacter, or Bordetella.

24. The method of claim 1 wherein the bacterial infection is caused by Streptococcus pneumoniae, Staphylococcus aureus or Haemophilus influenzae.
